SEQUENCE LISTING (25 sequences)

SEQ ID NO: 1 - protein, 619 aa, Arachis hypogaea
MRGRVSPLMLLLGILVLASVSATHAKSSPYQKKTENPCAQRCLQSCQQEPDDLKQKACESRCTKLEYDPRCVYDPRGHTGTTNQRSPPGERTRGRQPGDYDDDRRQPRREEGGRWGPAGPREREREEDWRQPREDWRRPSHQQPRKIRPEGREGEQEWGTPGSHVREETSRNNPFYFPSRRFSTRYGNQNGRIRVLQRFDQRSRQFQNLQNHRIVQIEAKPNTLVLPKHADADNILVIQQGQATVTVANGNNRRALILTRAMHSESHPFHFLHLDDMTPELRVAKSHAVNTPGQFEDFFPASSRDQSSYLQGFSRNTLEAAFNAEFNEIRRVLLEENAGGEQEERGQRRWSTRSSENNEGVIVEVSKEHVEELTKHAKSVSKKGSEEEGDITNPINLREGEPDLSDNFGRLFEVKPDKKNPQLQDLDMMLTCVEIKEGALMLPHFNSKAMVIVVINKGTGNLELVAVRKEQQQRGRREQEWEEEEEDEEEEGSNREVRRYTARLKEGDVFIMPAAHPVAINASSELHLLGFGINAENNHRIFLAGDKDNVVDQIEKQAKDLAFPGSGEQVEKLIKNQRESHFVSARPQSQSPSSPEKEDQEEENQGGKGPLLSILKAFN

SEQ ID NO: 2 - protein, 160 aa, Arachis hypogaea
MAKLTILVALALFLLAAHASARQQWELQGDRRCQSQLERANLRPCEQHLMQKIQRDEDSYERDPYSPSQDPYSPSPYDRRGAGSSQHQERCCNELNEFENNQRCMCEALQQIMENQSDRLQGRQQEQQFKRELRNLPQQCGLRAPQRCDLDVESGGRDRY

SEQ ID NO: 3 - protein, 512 aa, Arachis hypogaea
MAKLLALSLCFCVLVLGASSVTFRQGGEENECQFQRLNAQRPDNRIESEGGYIETWNPNNQEFQCAGVALSRTVLRRNALRRPFYSNAPLEIYVQQGSGYFGLIFPGCPSTYEEPAQEGRRYQSQKPSRRFQVGQDDPSQQQQDSHQKVHRFDEGDLIAVPTGVAFWMYNDEDTDVVTVTLSDTSSIHNQLDQFPRRFYLAGNQEQEFLRYQQQQGSRPHYRQISPRVRGDEQENEGSNIFSGFAQEFLQHAFQVDRQTVENLRGENEREEQGAIVTVKGGLRILSPDEEDESSRSPPNRREEFDEDRSRPQQRGKYDENRRGYKNGIEETICSASVKKNLGRSSNPDIYNPQAGSLRSVNELDLPILGWLGLSAQHGTIYRNAMFVPHYTLNAHTIVVALNGRAHVQVVDSNGNRVYDEELQEGHVLVVPQNFAVAAKAQSENYEYLAFKTDSRPSIANQAGENSIIDNLPEEVVANSYRLPREQARQLKNNNPFKFFVPPFDHQSMREVA

SEQ ID NO: 4 - protein, 507 aa, Arachis hypogaea
RQQPEENACQFQRLNAQRPDNRIESEGGYIETWNPNNQEFECAGVALSRLVLRRNALRRPFYSNAPQEIFIQQGRGYFGLIFPGCPRHYEEPHTQGRRSQSQRPPRRLQGEDQSQQQRDSHQKVHRFDEGDLIAVPTGVAFWLYNDHDTDVVAVSLTDTNNNDNQLDQFPRRFNLAGNTEQEFLRYQQQSRQSRRRSLPYSPYSPQSQPRQEEREFSPRGQHSRRERAGQEEENEGGNIFSGFTPEFLEQAFQVDDRQIVQNLRGETESEEEGAIVTVRGGLRILSPDRKRRADEEEEYDEDEYEYDEEDRRRGRGSRGRGNGIEETICTASAKKNIGRNRSPDIYNPQAGSLKTANDLNLLILRWLGPSAEYGNLYRNALFVAHYNTNAHSIIYRLRGRAHVQVVDSNGNRVYDEELQEGHVLVVPQNFAVAGKSQSENFEYVAFKTDSRPSIANLAGENSVIDNLPEEVVANSYGLQREQARQLKNNNPFKFFVPPSQQSPRAVA

SEQ ID NO: 5 - protein, 530 aa, Arachis hypogaea
MAKLLELSFCFCFLVLGASSISFRQQPEENACQFQRLNAQRPDNRIESEGGYIETWNPNNQEFECAGVALSRLVLRRNALRRPFYSNAPQEIFIQQGRGYFGLIFPGCPSTYEEPAQQGRRYQSQRPPRRLQEEDQSQQQQDSHQKVHRFNEGDLIAVPTGVAFWLYNDHDTDVVAVSLTDTNNNDNQLDQFPRRFNLAGNHEQEFLRYQQQSRQSRRRSLPYSPYSPHSRPRREEREFRPRGQHSRRERAGQEEEDEGGNIFSGFTPEFLEQAFQVDDRQIVQNLWGENESEEEGAIVTVRGGLRILSPDGTRGADEEEEYDEDQYEYHEQDGRRGRGSRGGGNGIEETICTACVKKNIGGNRSPHIYDPQRWFTQNCHDLNLLILRWLGLSAEYGNLYRNALFVPHYNTNAHSIIYALRGRAHVQVVDSNGNRVYDEELQEGHVLVVPQNFAVAGKSQSENFEYVAFKTDSRPSIANFAGENSFIDNLPEEVVANSYGLPREQARQLKNNNPFKFFVPPFQQSPRAVA

SEQ ID NO: 6 - protein, 131 aa, Arachis hypogaea
MSWQTYVDDHLLCEIEGNHLSSAAILGQDGSVWAQSSNFPQFKPEEITAIMNDFAEPGSLAPTGLYLGGTKYMVIQGEPGTVIRGKKGPGGVTIKKTNQALIIGIYDEPMTPGQCNMIVEKLGDYLIDTGL

SEQ ID NO: 7 - protein, 145 aa, Arachis hypogaea
MAKSTILVALLALVLVAHASAMRRERGRQGDSSSCERQVDRVNLKPCEQHIMQRIMGEQEQYDSYDIRSTRSSDQQQRCCDELNEMENTQRCMCEALQQIMENQCDRLQDRQMVQQFKRELMNLPQQCNFRAPQRCDLDVSGGRC

SEQ ID NO: 8 - protein, 164 aa, Arachis hypogaea
MVKLSILVALLGALLVVASATRWDPDRGSRGSRWDAPSRGDDQCQRQLQRANLRPCEEHIRQRVEKEQEQEQDEYPYIQRGSRGQRPGESDEDQEQRCCNELNRFQNNQRCMCQALQQILQNQSFRFQQDRSQLHQMERELRNLPQNCGFRSPSRCDLSSRTPY

SEQ ID NO: 9 - protein, 157 aa, Arachis hypogaea
MGVFTFEDEITSTVPPAKLYNAMKDADSITPKIIDDVKSVEIVEGNGGPGTIKKLTIVEDGETKFILHKVESIDEANYAYNYSVVGGVALPPTAEKITFETKLVEGPNGGSIGKLTLKYHTKGDAKPDEEELKKGKAKGEGLFRAIEGYVLANPTQY

SEQ ID NO: 10 - protein, 116 aa, Arachis hypogaea
MASLKFAFVMLVCMAMVGAPMVNAISCGQVNSALAPCIPFLTKGGAPPPACCSGVRGLLGALRTTADRQAACNCLKAAAGSLRGLNQGNAAALPGRCGVSIPYKISTSTNCATIKF

SEQ ID NO: 11 - protein, 92 aa, Arachis hypogaea
LSCGQVNSALAPCITFLTKGGVPSGPCCSGVRGLLGAAKTTADRQAACNCLKAAAGSLHGLNQGNAAALPGRCGVSIPYKISTSTNCATIKF

SEQ ID NO: 12 - protein, 169 aa, Arachis hypogaea
MTDRTQPHTVQVHTTAGRFGDTAAGTNRYPDRGPSTSKVIAVITGLPIGGTLLLFAGLALAGTLLGLAVTTPLFILFSPVIVPAIIVVGLSVAGFLTSGACGLTGLSSFSWVMNYIRQTHGSVPEQLEMAKHRMADVAGYVGQKTKDVGQKTKEVGQEIQTKAQDSKRT

SEQ ID NO: 13 - protein, 150 aa, Arachis hypogaea
MTDRTQPHAVQVHTTAGRFGDTAAGTNRYADRGPSTSKVIAVITGLPIGGTLLLFAGLALAGTLLGLAVTTPLFILFSPVIVPATIVVGLSVAGFLTSGACGLTGLSSFSWVMNYIRQTHGSVPEQLEMAKHRMADVAGYVGQKTKDVGQ

SEQ ID NO: 14 - protein, 137 aa, Arachis hypogaea
MAEALYYGGRQRQEQPRSTQLVKATTAVVAGGSLLILAGLVLAGTVIGLTTITPLFVIFSPVLVPAVITVALLGLGFLASGGFGVAAITVLTWIYRYVTGKHPPGANQLDTARHKLMGKAREIKDFGQQQTSGAQAS

SEQ ID NO: 15 - protein, 137 aa, Arachis hypogaea
MAEALYYGGRQRQDQPRSTQLVKATTAVVAGGSLLILAGLVLAATVIGLTTITPLFVIFSPVLVPAVITVALLGLGFLASGGFGVAAITVLTWIYRYVTGKHPPGANQLDTARHKLMSKAREIKDYGQQQTSGAQAS

SEQ ID NO: 16 - protein, 71 aa, Arachis hypogaea
KTVAGFCIFFLVLFLAQEGVVKTEAKLCNHLADTYRGPCFTNASCDDHCKNKEHFVSGTCMKMACWCAHNC

SEQ ID NO: 17 - protein, 79 aa, Arachis hypogaea
VQKRTIIMEKKMAGFCIFFLILFLAQEYGVEGKECLNLSDKFKGPCLGSKNCDHHCRDIEHLLSGVCRDDFRCWCNRKC

SEQ ID NO: 18 - protein, 72 aa, Arachis hypogaea
MEKKMAGFCIFFLVLFLAQEYGVEGKVCLNLSDKFKGPCLGTKNCDHHCRDIEHLLSGVCRDDFRCWCNRNC

SEQ ID NO: 19 - protein, 176 aa, Arachis hypogaea
MATATDRAPHQVQVHTPTTQRVDVPRRGYDVSGGGIKTLLPERGPSTSQIIAVLVGVPTGGTLLLLSGLSLLGTIIGLAIATPVFTFFSPVIVPAVVTIGLAVTGILTAGACGLTGLMSLSWMINFIRQVHGTTVPDQLDSVKRRMADMADYVGQKTKDAGQQIQTKAQDVKRSSS

SEQ ID NO: 20 - protein, 176 aa, Arachis hypogaea
MATATDRAPHQVQVHTPTTQRVDVQRRGYDVSGGGVKTFLPDRGPSTSQIIAVLVGVPTGGTLLLLSGLSLLGTIIGLAIATPVFTFFSPVIVPAVVTIGLAVIGILTAGACGLTGLMSLSWMINFIRQVHGTTVPDQLDSAKRRMADMADYVGQKTKDAGQEIQTKAQDVKRSSS

SEQ ID NO: 21 - protein, 176 aa, Arachis hypogaea
MATATDRAPHQVQVHTPTTQRVDVPRRGYDVSGGGIKTLLPERGPSTSQIIAVLVGVPTGGTLLLLSGLSLLGTIIGLAIATPVFIFFSPVIVPAVVTIGLAVTGILTAGACGLTGLMSLSWMINFIRQVHGTTVPDQLDSVKRRMADMADYVGQKTKDAGQEIQTKAQDVKRSSS

SEQ ID NO: 22 - protein, 166 aa, Arachis hypogaea
MSDQTRTGYGGGGSYGSSYGGGGTYGSSYGTSYDPSTNQPIRQAIKFMTASTIGVSFLILSGLILTGTVIGLIIATPLLVIFSPILVPAAITLALAAGGFLFSGGCGVAAIAALSWLYSYVTGKHPAGSDRLDYAKGVIADKARDVKDRAKDYAGAGRAQEGTPGY

SEQ ID NO: 23 - protein, 68 aa, Arachis hypogaea
VTCTPTELSPCLGAITGGSPPSSVCCQKLRAQKPCLCNYIKNPALRTYVNSPGARRVASSCGVPLPSC

SEQ ID NO: 24 - protein, 93 aa, Arachis hypogaea
AIQCSFVTKSIAPCFGYLKSGGTVSGPCCSGIQNINGTAKTTSDRQAVCNCLKSAAASLGSQINPNNAASLPGKCGVSIPYKISTSINCSSIK

SEQ ID NO: 25 - protein, 226 aa, Arachis hypogaea
MANPRVFFDMTIGGQPAGRIIMELFADTTPRTAENFRALCTGEKGVGRSGKPLHYKGSSFHRVIPNFMCQGGDFTAGNGTGGESIYGSKFADENFIKKHTGPGILSMANAGQNTNGSQFFICTAKTEWLDGKHVVFGQVVEGLDVVREIEKVGSSSGRTSKPVVVADCGQLCNAKLTMSWGNGAYRKSLQVQGVVIGQRDANSMSEQAQFAFVVSFVTQIMKIQSS

(19) United States
(12) Patent Application Publication
(10) Pub. No.: US 2024/0201187 A1
(43) Pub. Date: Jun. 20, 2024

(54) Method of Tracking Maintenance of Immunological Tolerance

(71) Applicant: COUR PHARMACEUTICALS DEVELOPMENT COMPANY INC., Skokie, IL (US)

(72) Inventors: John Puisis, Skokie, IL (US); James Herrmann, Skokie, IL (US); Michael Boyne, Skokie, IL (US); Adam Elhofy, Chicago, IL (US); Joseph Podojil, Skokie, IL (US); Greta Wodarcyk, Skokie, IL (US)

(21) Appl. No.: 18/287,065

(22) PCT Filed: Apr. 15, 2022
(86) PCT No.: PCT/US22/24955

§ 371 Date: Oct. 16, 2023

Related U.S. Application Data
(60) Provisional application No. 63/175,973, filed on Apr. 16, 2021.

(51) Int. Cl. G01N 33/569; G01N 33/68

(52) U.S. Cl. CPC G01N 33/56972; G01N 33/6863; G01N 2333/52; G01N 2800/24; G01N 2800/52

(57) ABSTRACT

The present application relates, in general, to methods for tracking the induction of and long-term maintenance of antigen specific immune tolerance during administration of an antigen specific immune tolerizing therapy, such as a TIMP.

### CROSS-REFERENCE TO RELATED APPLICATIONS

The present application claims the priority benefit of U.S. Provisional Patent Application No. 63/175,973, filed Apr. 16, 2021, hereby incorporated by reference in its entirety.

### FIELD OF THE DISCLOSURE

The present disclosure relates, in general, to methods of monitoring and tracking tolerance induction and long-term maintenance of tolerance in a subject after receiving tolerizing nanoparticle therapy.

### BACKGROUND

Several inflammatory diseases and conditions are characterized by excessive, abnormal, and/or dysregulated immune responses to antigens like self-antigens (e.g., autoimmune diseases), therapeutic proteins, and allergens (e.g., food allergens and environmental allergens). Dysregulated functioning of innate (e.g., monocytes, macrophages, neutrophils, dendritic cells, and microglia) and adaptive immune cells (e.g., T-cells, B-cells, and NK cells) is a major driver of the excessive and abnormal immune response to these antigens. Activation and migration of immune cells to sites of inflammation, abnormal production of cytokines, chemokines, antibodies and other factors, changes in cellular metabolism, and altered angiogenic responses are among the critical processes that contribute to pathology in several inflammatory diseases and conditions. Conventional approaches for treating several inflammatory diseases and conditions rely on the chronic use of broadly immunosuppressive drugs that do not address the root cause of disease, provide only symptomatic relief, and cause significant side-effects such increased risks of infection, malignancies, organ failure, and even death in some cases.

Induction of antigen-specific immune tolerance (ASIT) has been postulated as a gold-standard for the treatment of several inflammatory conditions. ASIT relies on reprogramming the immune system to rebuild tolerance to disease-specific antigens implicated in pathology.

Several attempts at inducing antigen-specific immune tolerance have been made and have been described previously (see International Patent Publications WO2009/056332; WO2009/056833A2; WO2010/060155A1; WO2013/184976A2; WO2014159609); however, the clinical translation has proven challenging due to lack of efficacy in humans, safety concerns, and complex manufacturing. At best, these therapies have been able to achieve desensitization to the antigen but not true immune tolerance.

Often confused, desensitization and true immune tolerance are achieved via different immunological mechanisms and have distinct outcomes. Desensitization is achieved by administration of increasing doses of antigens over a long period of time up to a tolerated maximum dose. Importantly, desensitizing therapies protect against accidental exposure and require chronic administration such that the desensitizing effect is lost as soon as the therapy is stopped. By contrast, tolerance induction facilitates immune reprogramming leading to modulation of the physiological immune response to an antigen or allergen, for example by modifying cytokine secretion or modifying a T cell response. Recently, polymeric nanoparticles encapsulating disease-associated antigens, called Tolerizing Immune Modifying Particles (TIMPs) have been described for inducing true antigen specific immune tolerance (ASIT) (See WO/2013/1952532A2 and WO/2015/023796A2). TIMPs encapsulating gliadin, the offending antigen in Celiac Disease (CD), demonstrated efficacy at inducing tolerance to gliadin in a Phase 2 clinical trial in CD subjects. Efficacy in this clinical study was established in CD subjects receiving TIMPs by assaying inflammatory immune responses to gluten ingestion following treatment. Thus, TIMPs go beyond desensitization by protecting against much higher levels of exposure to the offending antigen.

Existing analytical methods are not well-suited for assessing induction of true immune tolerance and are geared towards assessing induction of desensitization instead (WO2012148549A; WO2019028028). These approaches are incapable of providing deeper insights into the status of immune tolerance which could enable therapeutic decisions due to their reliance on assaying a limited, if not just one, set of parameters (e.g., a single cell type or a limited panel of cytokines/chemokines). A drawback of current approaches is their ability to provide an assessment of the immune system only at the specific time the sample was collected from the subject for analysis and not a comprehensive view of the status of true immune tolerance.

### SUMMARY OF THE INVENTION

An important factor influencing the success of ASIT is the longevity of immune tolerance. Several physiological and immunological factors determine the longevity of immune tolerance induced by therapeutic intervention and immune tolerance may begin to diminish over time. It is therefore important to routinely monitor subjects treated with tolerizing therapies to confirm the maintenance of immunological tolerance and re-dose the subject with the tolerizing therapy if a change, weakening, or loss of immune tolerance is observed. However, to our knowledge, there are no existing methods for monitoring maintenance of immunological tolerance in a subject treated with a therapy capable of inducing true immune tolerance and which help inform re-dosing decisions to ensure maintenance of antigen-specific immune tolerance to prevent disease relapse.

In various embodiments, the present disclosure provides methods for monitoring the immune tolerance status of a subject undergoing treatment for an inflammatory disease or condition and determining whether the subject requires re-administration of the treatment. In various embodiments, the method comprises the steps of (a) obtaining one or more biological samples from the subject prior to administration of treatment and determining the immune tolerance status of the subject by assaying said biological sample(s), (b) obtaining one or more biological samples from the subject after administration of treatment and determining the immune tolerance status of the subject by assaying the biological sample(s), (c) obtaining one or more biological samples from the subject at regular intervals after administration of treatment and determining the immune tolerance status of the subject by assaying the biological sample(s), and (d) re-administering treatment if the immune tolerance status determined in step (c) indicates a change, weakening, and/or loss of immunological tolerance. In various embodiments, the results from the assay of one or more biological samples in step (c) are compared to the results from the assay of one or more biological samples in steps (a) and/or (b) to generate a signature of immune tolerance status.

In various embodiments, the biological sample collected from the subject is whole-blood, peripheral blood, peripheral blood mononuclear cells (PBMCs), serum, plasma, urine, cerebrospinal fluid (CSF), stool, a tissue biopsy, and/or a bone-marrow biopsy. In various embodiments, the assay of the biological sample(s) consists of analyzing levels of, and or presence or absence of cell-surface proteins, extracellular proteins, intracellular proteins, nucleic acids, metabolites, and/or combinations thereof. In various embodiments, the assay of one or more biological sample(s) is used to generate a signature of immune tolerance status.

In various embodiments, the one or more biological samples of step (a) are collected from the subject 1-7 days, 1-4 weeks, and/or 1-12 months prior to administration of immune tolerizing therapy. In various embodiments, the one or more biological samples of step (b) are collected 1-7 days, 1-4 weeks, and/or 1-12 months after administration of the immune tolerizing therapy. In various embodiments, the one or more biological samples of step (c) are collected every 1-7 days, every 1-4 weeks, and/or every 1-12 months after administration of the immune tolerizing therapy. In various embodiments, the one or more biological samples of step (c) are collected at intervals of 1-7 days, every 1-4 weeks, and/or every 1-12 months after administration of the immune tolerizing therapy. In various embodiments, the samples are collected every week, 2 weeks, 4 weeks, 1 month, 2 months, 3 months, 4 months, 5 months, 6 months, 9 months or 12 months.

In various embodiments, the disclosure provides methods of monitoring immune tolerance. In various embodiments, the subject is undergoing treatment with an immune tolerizing therapy. In various embodiments, the subject is undergoing treatment with a desensitization therapy. In various embodiments, the immune tolerizing therapy is antigen specific. In various embodiments, the treatment administered to the subject is selected from the group consisting of oral immunotherapy (OIT), subcutaneous immunotherapy (SCIT), sublingual immunotherapy (SLIT), and immune tolerizing nanomedicine. In various embodiments, the treatment is an immune tolerizing nanomedicine. In various embodiments, the immune tolerizing nanomedicine consists of tolerizing immune modulating particles (TIMPs).

In various embodiments, the antigen-specific immune tolerizing therapy is a nanomedicine. In various embodiments, the nanomedicine consists of particles coupled to and/or encapsulating one or more antigens. In various embodiments, the particles are biodegradable. In various embodiments, the particles are made from metals selected from the group consisting of iron, iron oxide, gold, zinc, cadmium, and silver. In various embodiments, the particles have a negative zeta potential. In various embodiments, the zeta potential of the particle is from about −100 mV to about 0 mV. In various embodiments, the zeta potential of the particles is from about −100 mV to about −30 mV, from about −80 mV to about −30 mV, from about −75 mV to about −35 mV, from about −70 mV to about −30 mV, from about −60 mV to about −35 mV, or from about −50 mV to about −30 mV. In various embodiments, the zeta potential is about −30 mV, −35 mV, −40 mV, −45 mV, −50 mV, −55 mV, −60 mV, −65 mV, −70 mV, −75 mV, −80 mV, −85 mV, −90 mV, −95 mV or −100 mV.

In various embodiments, the diameter of the particle is between about 0.02 and 10 μm. In various embodiments, the diameter of the particle is between about 0.05 and 10 μm. In various embodiments, the diameter of the particle is between about 0.1 and 5 μm. In various embodiments, the diameter of the particle is between 0.2 μm and about 2 μm. In various embodiments, the diameter of the particle is between 0.2 μm and about 2 μm. In various embodiments, the diameter of the particle is between about 0.3 μm to about 5 μm. In various embodiments, the diameter of the particle is between about 0.5 μm to about 3 μm. In various embodiments, the diameter of the particle is between about 0.5 μm to about 1 μm. In various embodiments, the diameter of the particle is between about 20 to 10000 nm, 50 to 10000 nm, 50 to 5000 nm, 50 to 2000 nm, 100 to 1500 nm, about 300 to 1000 nm, about 400 to 800 nm or about 200 to 700 nm. In various embodiments, the diameter of the particle has an average diameter is about 20 nm, 50 nm, 100 nm, 200 nm, 300 nm, 400 nm, 500 nm, 600 nm, 700 nm, 800 nm, 900 nm, 1000 nm, 1100 nm, 1200 nm, 1300 nm, 1400 nm, 1500 nm, or 2000 nm. In various embodiments, the diameter of the negatively charged particle is between 300 nm to 800 nm.

In various embodiments, TIMPs consist of particles encapsulating one or more antigens. In various embodiments, the antigen is an autoimmune antigen, a transplant antigen, an allergen, an enzyme replacement therapy, a protein therapeutic, and/or a gene therapy vector or viral vector.

In various embodiments, TIMPs encapsulate one or more antigens selected from the group consisting of myelin basic protein, acetylcholine receptor, endogenous antigen, myelin oligodendrocyte glycoprotein (MOG), myelin basic protein (MBP), proteolipid protein (PLP), myelin associated glycoprotein (MAG), cyclic nucleotide phosphohydrolase, pancreatic beta-cell antigen, insulin, proinsulin, islet-specific glucose-6-phophatase catalytic subunit-related protein (IGRP), glutamic acid decarboxylase (GAD), collagen type 11, human cartilage gp39, fp130-RAPS, fibrillarin, small nucleolar protein, thyroid stimulating factor receptor, histones, glycoprotein gp70, pyruvate dehydrogenase dehydrolipoamide acetyltransferase (PCD-E2), hair follicle antigen, aqua porin 4, Desmoglein 1, Desmoglein 3, nicotinic acetylcholine receptor, gliadin, ADAMTS13, GPllb/GPIlla, CYP2D6, BP180, NC16, BP230, Ro60, MPO, thyroid stimulating hormone receptor, and human tropomyosin isoform 5, Bahia grass pollen (BaGP), peach allergen, milk allergens, celery allergens, nut allergens, tree-nut allergen, bovine serum albumin, Hazelnut allergens, ovalbumin, egg allergen, peanut allergens, fish allergens, shellfish allergens, dust mite, cat allergen, dog allergen, pollen allergen, bee venom, Japanese cedar pollen, an enzyme replacement therapy, a therapeutic protein, and a viral vector.

In various embodiments, the peanut allergen is selected from the group consisting of Ara h1, Ara h2, Ara h3, Ara h5, Ara h6, Ara h7, and Ara h8. In various embodiments, the peanut allergen is selected from the group consisting of Ara h1, Ara h2, Ara h3, Ara h4, Ara h5, Ara h6, Ara h7, Ara h8, Ara h9, Ara h10, Ara h11, Ara h12, Ara h13, Ara h14, Ara h15, Ara h16, and Ara h17.

In various embodiments, the antigen is an enzyme replacement therapy selected from the group consisting of Agalsidase beta, Agalsidase alfa, Imiglucirase, Taliglucirase alfa, Velaglucerase alfa, Alglucerase, Sebelipase alpha, Laronidase, Idursulfase, Elosulfase alpha, Galsulfase, Alglucosidase alpha, Factor VII, Factor VIII, Factor IX, Acetylgalactosamine 4-sulfate, Iduronidase, Alglucerase, Glucocerebrosidase.

In various embodiments, the protein therapeutic is a recombinant protein selected from the group consisting of erythropoietin, insulin, human growth hormone, follicle-stimulating hormone, granulocyte colony-stimulating factor, tissue plasminogen activator, insulin-like growth factor, uricase, kynurinase, L-arginine deaminase, arginase, methionine-γ-lyase, asparaginase, an amino acid degrading enzyme, a gluten degrading enzyme, a nucleotide degrading enzyme, IFN-γ, IL-2, IL-12, and IL-15.

In various embodiments, the protein therapeutic is an antibody. In various embodiments, the antibody is a monoclonal antibody or a polyclonal antibody. In various embodiments, the antibody is mono-specific, bi-specific, tri-specific, or bi-specific T-cell engager. In various embodiments the antibody targets receptor tyrosine kinase (RTK), EGFR, VEGF, VEGFR, PDGF, PDGFR, HER2/Neu, ER, PR, TGF-β1, TGF-β2, TGF-ß3, SIRP-α, PD-1, PD-L1, CTLA-4, CD3, CD25, CD19, CD20, CD39, CD47, CD73, FAP, IL-1B, IL-12, IL-2R, IL-15, IL-15R, IL-23, IL-33, IL-2R, IL-4Rα, T-cells, B-cells, NK cells, macrophages, monocytes, and/or neutrophils. In various embodiments, the antibody is selected from the group consisting of abciximab, adalimumab, alemtuzumab, avelumab, azetolizumab, basiliximab, bevacizumab, bezlotoxumab, blinatumomab, canakinumab, certolizumab, cetuximab, daclizumab, denosumab, durvalumab, efalizumab, emicizumab, etokimab, golimumab, ipilimumab, ixekizumab, infliximab, natalizumab, nivolumab, olaratumab, omalizumab, ofatimumab, palivizumab, panitumumab, pembrolizumab, ramucirumab, rituximab, tocilizumab, trastuzumab, tremelimumab, secukinumab, ustekinumab, and vedolizumab.

In various embodiments, the viral vector is selected from the group consisting of adenovirus, adeno-associated virus (AAV), herpes simplex virus, lentivirus, retrovirus, alphavirus, flavivirus, rhabdovirus, measles virus, Newcastle disease virus, poxvirus, vaccinia virus, modified Ankara virus, vesicular stomatitis virus, picornavirus, tobacco mosaic virus, potato virus x, comovirus or cucumber mosaic virus. In various embodiments, the virus is an oncolytic virus. In various embodiments the virus is a chimeric virus, a synthetic virus, a mosaic virus or a pseudotyped virus. In various embodiments, the AAV vector is AAV1, AAV2, AAV3, AAV4, AAV5, AAV6, AAV7, AAV8, AAV9, AAV12, Anc80 or combinations thereof.

In various embodiments, TIMPs are biodegradable. In various embodiments, the TIMPs are made from polymers selected from the group consisting of polylactic acid (PLA), polyglycolic acid (PGA), poly (lactic co-glycolic acid) (PLGA), polystyrene, a liposome, a lipid, PEG, cyclodextran, chitosan, and polysaccharides.

In various embodiments, TIMPs are surface functionalized. In various embodiments, TIMPs are surface functionalized by carboxylation. In various embodiments, TIMPs have a negative zeta potential. In various embodiments, the zeta potential of the particle is from about −100 mV to about 0 mV. In various embodiments, the zeta potential of the particle is from about −100 mV to about −30 mV, from about −80 mV to about −30 mV, from about −75 mV to about −35 mV, from about −70 mV to about −30 mV, from about −60 mV to about −35 mV, or from about −50 mV to about −30 mV. In various embodiments, the zeta potential is about −30 mV, −35 mV, −40 mV, −45 mV, −50 mV, −55 mV, −60 mV, −65 mV, −70 mV, −75 mV, −80 mV, −85 mV, −90 mV, −95 mV or −100 mV. In various embodiments, the diameter of TIMPs is between about 0.05 μm to about 10 μm. In various embodiments, the diameter of TIMPs is between 0.1 μm and about 10 μm. In various embodiments, the diameter of TIMPs is between 0.1 μm and about 5 μm. In various embodiments, the diameter of TIMPs is between 0.1 μm and about 3 μm. In various embodiments, the diameter of TIMPs is between 0.3 μm and about 5 μm. In various embodiments, the diameter of TIMPs is about 0.3 μm to about 3 μm. In various embodiments, the diameter of TIMPs is between about 0.3 μm to about 1 μm. In various embodiments, the diameter of TIMPs is between about 0.4 μm to about 1 μm. In various embodiments, the particle has a diameter of about 100 to 10000 nm, about 100 to 5000 nm, about 100 to 3000 nm, about 100 to 2000 nm, about 300 to 5000 nm, about 300 to 3000 nm, about 300 to 1000 nm, about 300 to 800 nm, about 400 to 800 nm, or about 200 to 700 nm. In various embodiments, the diameter of TIMPs is about 50 nm, 100 nm, 200 nm, 300 nm, 400 nm, 500 nm, 600 nm, 700 nm, 800 nm, 900 nm, 1000 nm, 1100 nm, 1200 nm, 1300 nm, 1400 nm, 1500 nm, or 2000 nm. In various embodiments, the diameter of the negatively charged particle is between 400 nm to 800 nm. TIMPs have been described in WO/2013/1952532A2 and WO/2015/023796A2, which are incorporated herein by reference.

In various embodiments, the present invention provides methods for monitoring the immune tolerance status of a subject undergoing treatment for an inflammatory disease or condition and determining whether the subject requires re-administration of the treatment. In various embodiments, the present invention provides methods for monitoring whether a subject treated with an antigen-specific immune tolerizing therapy has maintained immunological tolerance, and whether the subject requires re-administration of the antigen-specific immune tolerizing therapy. In various embodiments, the method comprises the steps of (a) obtaining one or more biological samples from a subject prior to administration of antigen-specific immune tolerizing therapy and determining the immune tolerance status of the subject by assaying the biological sample(s), (b) obtaining one or more biological samples from a subject after administration of the antigen-specific immune tolerizing therapy and determining the immune tolerance status of the subject by assaying the biological sample(s), (c) obtaining one or more biological samples from the subject at regular intervals after administration of the antigen-specific immune tolerizing therapy and determining the immune tolerance status of the subject by assaying the biological sample(s), and (d) re-administering the antigen-specific immune tolerizing therapy if the immune tolerance status determined in step (c) indicates a change, weakening, and/or loss of immune tolerance. In various embodiments, the immune tolerance status determined in step (c) is compared to the immune tolerance status determined in steps (a) and/or (b). In various embodiments, the results from the assay of one or more biological samples in Step (c) are compared to the results from the assay of one or more biological samples in Steps (a) and/or (b) to generate a signature of immune tolerance status. In various embodiments, the assay of the biological sample(s) consists of the analysis of cell-surface proteins, extracellular proteins, intracellular proteins, nucleic acids, and/or combinations thereof. In various embodiments, the assay of one or more biological samples described in Steps (a)-(c) is used to generate an immune tolerance signature.

Also provided is a method for monitoring the tolerance status of a subject undergoing tolerizing treatment for an inflammatory disease or condition, the method comprising the steps of: (a) obtaining one or more biological samples from the subject prior to administration of treatment and determining the immune tolerance status of the subject by assaying said biological sample(s), (b) obtaining one or more biological samples from the subject after administration of treatment and determining the immune tolerance status of the subject by assaying the biological sample(s), and (c) obtaining one or more biological samples from the subject at regular intervals after administration of treatment and determining the immune tolerance status of the subject by assaying the biological sample(s), (d) comparing the results from the assay of one or more biological samples in step (c) with the results of steps (a) and/or (b) to generate an immune tolerance signature, and (e) re-administering the tolerizing treatment if the immune tolerance signature indicates a weakening, and/or loss of immunological tolerance.

In various embodiments, the change, weakening, and/or loss of immune tolerance is determined by comparing the results from the assay of one or more biological samples in step (c) to the results from steps (a) and/or (b). In various embodiments, the comparison of results from step (c) to the results from steps (a) and/or (b) indicates weakening and/or loss of immune tolerance by about 5%-100% (e.g., about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, inclusive of all values and ranges between these values), 10-95%, 15-90%, 20-85%, 25-75%, 30-70%, 35-65%, 40-60%, 45-55%, or 50%. In various embodiments, the comparison of results from step (c) to the results from steps (a) and/or (b) indicates weakening and/or loss of immune tolerance by about 2-100-fold (e.g., about 2, 5, 10, 15, 20, 25, 30, 35, 40, 45, 50, 55, 60, 65, 70, 75, 80, 85, 90, 95, or 100-fold, inclusive of all values and ranges between these values). In various embodiments, the subject is re-administered the immune tolerizing therapy in step (d) if the comparison of results from step (c) to the results from steps (a) and/or (b) indicates weakening and/or loss of immune tolerance by about >5% (e.g., about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, inclusive of all values and ranges between these values). In various embodiments, the subject is re-administered the immune tolerizing therapy in step (d) or (e) if the comparison of results from the assay of one or more biological samples in step (c) to the results from Steps (a) and/or (b) indicates weakening and/or loss of immune tolerance by about >2-fold (e.g., about 2, 5, 10, 15, 20, 25, 30, 35, 40, 45, 50, 55, 60, 65, 70, 75, 80, 85, 90, 95, or 100-fold, inclusive of all values and ranges between these values).

In various embodiments, the present disclosure provides methods for monitoring the immune tolerance status of a subject undergoing treatment for an inflammatory disease or condition and determining whether the subject requires re-administration of the treatment. In various embodiments, the present disclosure provides methods for monitoring whether a subject treated with an antigen-specific immune tolerizing therapy has maintained immune tolerance, and whether the subject requires re-administration of the antigen-specific immune tolerizing therapy. In various embodiments, the antigen-specific immune tolerizing therapy comprises administering to the subject an effective amount of TIMPs. In various embodiments, the method comprises the steps of (a) obtaining one or more biological samples from a subject prior to administration of TIMPs and determining the immune tolerance status of the subject by assaying the biological sample(s), (b) obtaining one or more biological samples from a subject after administration of TIMPs and determining the immune tolerance status of the subject by assaying the biological sample(s), (c) obtaining one or more biological samples from the subject at regular intervals after administration of TIMPs and determining the immune tolerance status of the subject by assaying the biological sample(s), and (d) re-administering TIMPs if the immune tolerance status determined in step (c) indicates a change, weakening, and/or loss of immune tolerance. In various embodiments, the results from the assay of one or more biological samples in step (c) are compared to the results from the assay of one or more biological samples in steps (a) and/or (b) to generate a signature of immune tolerance status. In various embodiments, the assay of the biological sample(s) is selected form the group consisting of analyzing cell-surface proteins, extracellular proteins, intracellular proteins, nucleic acids, and combinations thereof. In various embodiments, the assay of biological samples described in steps (a)-(c) is used to generate an immune tolerance signature.

In various embodiments, the one or more biological samples of step (a) are collected from the subject 1-7 days, 1-4 weeks, and/or 1-12 months prior to administration of TIMPs. In various embodiments, the one or more biological samples of step (b) are collected 1-7 days, 1-4 weeks, and/or 1-12 months after administration of TIMPs. In various embodiments, the one or more biological samples of step (c) are collected every 1-7 days, every 1-4 weeks, and/or every 1-12 months after administration of TIMPs. In various embodiments, the one or more biological samples of step (c) are collected at intervals of 1-7 days, every 1-4 weeks, and/or every 1-12 months after administration of the immune tolerizing therapy.

In various embodiments, the change, weakening, and/or loss of immune tolerance is determined by comparing the results from the assay of one or more biological samples in Step (c) to the results from Steps (a) and/or (b). In various embodiments, the comparison of results from Step (c) to the results from Steps (a) and/or (b) indicates weakening and/or loss of immune tolerance by about 5%-100% (e.g., about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, inclusive of all values and ranges between these values), 10-95%, 15-90%, 20-85%, 25-75%, 30-70%, 35-65%, 40-60%, 45-55%, or 50%. In various embodiments, the comparison of results from Step (c) to the results from Steps (a) and/or (b) indicates weakening and/or loss of immune tolerance by about 2-100-fold (e.g., about 2, 5, 10, 15, 20, 25, 30, 35, 40, 45, 50, 55, 60, 65, 70, 75, 80, 85, 90, 95, or 100-fold, inclusive of all values and ranges between these values). In various embodiments, the subject is re-administered TIMPs in Step (d) if the comparison of results from Step (c) to the results from Steps (a) and/or (b) indicates weakening and/or loss of immune tolerance by about >5% (e.g., about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, inclusive of all values and ranges between these values).

In various embodiments, the subject is re-administered TIMPs if the comparison of results from the assay of one or more biological samples in Step (c) to the results from Steps (a) and/or (b) indicates weakening and/or loss of immune tolerance by about >2-fold (e.g., about 2, 5, 10, 15, 20, 25, 30, 35, 40, 45, 50, 55, 60, 65, 70, 75, 80, 85, 90, 95, or 100-fold, inclusive of all values and ranges between these values). In various embodiments, the TIMPs are administered at a dose level of between about 0.1 mg/kg to 12 mg/kg. In various embodiments, the TIMPs are administered at a dose level of 0.1 mg/kg, 0.25 mg/kg, 0.5 mg/kg, 0.75 mg/kg, 1.0 mg/kg, 1.25 mg/kg, 1.5 mg/kg, 2.0 mg/kg, 2.5 mg/kg, 3 mg/kg, 3.5 mg/kg, 4.0 mg/kg, 4.5 mg/kg, 5 mg/kg, 5.5 mg/kg, 6 mg/kg, 6.5 mg/kg, 7 mg/kg, 7.5 mg/kg, 8.0 mg/kg, 8.5 mg/kg, 9 mg/kg, 9.5 mg/kg, 10 mg/kg, 10.5 mg/kg, 11 mg/kg, 11.5 mg/kg, or 12 mg/kg. In various embodiments, the TIMPs are administered at a dose level of about 50 mg, 75 mg, 100 mg, 125 mg, 150 mg, 175 mg, 200 mg, 225 mg, 250 mg, 275 mg, 300 mg, 325 mg, 350 mg, 375 mg, 400 mg, 425 mg, 450 mg, 500 mg, 525 mg, 550 mg, 575 mg, 600 mg, 625 mg, 650 mg, 675 mg, 700 mg, 725 mg, 750 mg, 775 mg, or 800 mg. In various embodiments, TIMPs are administered at a concentration of 0.05 mg/mL, 0.1 mg/mL, 0.5 mg/mL, 1 mg/mL, 2 mg/mL, 3 mg/mL, 4 mg/mL, 5 mg/mL, 6 mg/mL, 7 mg/mL, 8 mg/mL, 9 mg/mL, 10 mg/mL, 11 mg/mL, 12.5 mg/mL, 15 mg/mL, 17.5 mg/mL, 20 mg/mL, 25 mg/mL, 30 mg/mL, 40 mg/mL, or 50 mg/mL.

In various embodiments, TIMPs are administered intravenously, subcutaneously, intramuscularly, intraperitoneally, intranasally, via inhalation or orally. In various embodiments, TIMPs are administered a single dose or in multiple doses. In various embodiments, TIMPs are administered in two doses one-week apart. In various embodiments, TIMPS are administered once weekly, once every two weeks, once every three weeks, once every 4 weeks, once every two months, once every three months, once every 6 months, or once per year.

In various embodiments, TIMPs are administered alone or in combination with one or more additional therapeutics. In various embodiments, the additional therapeutic is an inhibitor of IgE, an inhibitor of basophil activation, an inhibitor of mast cell activation, an antihistamine, or a small molecule or biological therapeutic. In various embodiments, the additional therapeutic inhibits IgE. In various embodiments, the additional therapeutic inhibits basophil activation. In various embodiments, the additional therapeutic inhibits mast cell activation. In various embodiments, the additional therapeutic is a biologic or a small molecule. In various embodiments, the additional therapeutic is an anti-IgE antibody, an anti-IL-4Rα antibody, an anti-IL13 antibody, an anti-IL-33 antibody, an antihistamine, a steroid, a corticosteroid, a leukotriene modifier, or an nonsteroid anti-inflammatory drug (NSAID).

In various embodiments, the additional therapeutic is an antihistamine. In various embodiments, the antihistamine is a first generation antihistamine. In various embodiments, the antihistamine is a second generation antihistamine. In various embodiments, the antihistamines are selected from the group consisting of brompheniramine, carbinoxamine maleate, chlorpheniramine, clemastine, diphenhydramine, hydroxyzine, triprolidine, azelastine, cetirizine, desloratadine, fexofenadine, levocetrizine, doxylamine, ebastine, embramine, epinephrine, fexofenadine, loratadine, and olopatadine.

In various embodiments, the additional therapeutic is a steroid. In various embodiments, the steroid is selected from the group consisting of beclomethasone, ciclesonide, fluticasone furoatr, mometasone, budenoside, fluticasone, triamcinolone, and loteprednol.

In various embodiments, the additional therapeutic is a corticosteroid. In various embodiments, the corticosteroid is selected from the group consisting of cortisone, prednisone, prednisolone, methylprednisolone, dexamethasone, betamethasone, and hydrocortisone.

In various embodiments, the additional therapeutic is a nonsteroid anti-inflammatory drug (NSAID). In various embodiments the NSAID is a non-selective NSAID. In various embodiments the NSAID is a COX-2 selective NSAID. In various embodiments the NSAID is a COX-1 selective NSAID. In various embodiments the NSAID is a prostaglandin synthase inhibitor. In various embodiments, the NSAID is selected from the group consisting of diclofenac, diclofenac potassium, diclofenac sodium, diflunisal, etodolac, flurbiprofen, fenoprofen, fenoprofen calcium, ketorolac, ketorolac tromethamine, ketoprofen, tolmetin, tolmetin sodium, aspirin, ibuprofen, naproxen, indomethacin, indomethacin sodium, sulindac, felbinac, piroxicam, mefenamic acid, meclofenamate sodium, meloxicam, nabumetone, oxaprozin, piroxicam, celecoxib, etodolac, etoricoxib, lumiracoxib, rofecoxib, and valdecoxib.

In various embodiments, the additional therapeutic is a leukotriene modifier. In various embodiments the leukotriene modifier is an antileukotriene. In various embodiments the leukotriene modifier is a leukotriene receptor antagonist. In various embodiments the leukotriene modifier is a leukotriene synthesis inhibitor. In various embodiments the leukotriene modifier is selected from the group consisting of montelukast, zileuton, and zafirlukast.

In various embodiments, the subject's immune tolerance status is determined by assaying one or more cells from the biological sample(s). In various embodiments, the cells assayed from the biological sample(s) are immune cells, non-immune cells, and/or combinations thereof. In various embodiments, the cells assayed from the biological sample(s) are immune cells. In various embodiments, the immune cells assayed from the biological sample(s) are innate immune cells, adaptive immune cells, and/or combinations thereof. In various embodiments the immune cells assayed from the biological sample(s) are innate immune cells. In various embodiments the immune cells assayed from the biological sample(s) are adaptive immune cells. In various embodiments the innate immune cells assayed from the biological sample(s) are antigen-presenting cells (APCs). In various embodiments, the innate immune cells assayed from the biological sample are monocytes, macrophages, neutrophils, granulocytes, dendritic cells, mast cells, eosinophils, basophils, and/or combinations thereof. In various embodiments, the adaptive immune cells assayed from the biological sample(s) are effector immune cells. In various embodiments, the adaptive immune cells assayed from the biological sample(s) are T-cells, B-cells, NK cells, NK-T cells, and/or combinations thereof. In various embodiments, the T cells are effector T cell, Th1 cells, Th2a cells, regulatory T cells (Treg), or Type 1 regulatory T cells (Tr1).

In various embodiments, the cells assayed from the biological sample(s) are epithelial cells, stromal cells, endothelial cells, fibroblasts, pericytes, adipocytes, mesenchymal stem cells, hematopoietic stem cells, hematopoietic progenitor cells, liver sinusoidal endothelial cells (LSECs), and/or Kupffer cells. In various embodiments, the assay of one or more cells from the biological sample(s) is used to generate a signature of immune tolerance status.

In various embodiments, the subject's immune tolerance status is determined by analyzing one or more cell-surface proteins from the biological sample(s). In various embodiments, the cell-surface proteins are selected from the group consisting of CD1c, CD2, CD3, CD4, CD5, CD8, CD9, CD10, CD11b, CD11c, CD14, CD15, CD16, CD18, CD19, CD20, CD21, CD22, CD23, CD24, TACI, CD25, CD27, CD28, CD30, CD30L, CD31, CD32, CD32b, CD34, CD33, CD38, CD39, CD40, CD40-L, CD41b, CD42a, CD42b, CD43, CD44, CD45, CD45RA, CD47, CD45RA, CD45RO, CD48, CD52, CD55, CD56, CD58, CD61, CD66b, CD69, CD70, CD72, CD79, CD68, CD84, CD86, CD93, CD94, CD95, CRACC, BLAME, BCMA, CD103, CD107, CD112, CD120a, CD120b, CD123, CD125, CD127, CD134, CD135, CD140a, CD141, CD154, CD155, CD160, CD161, CD163,CD172a, XCR1, CD203c, CD204, CD206, CD207 CD226, CD244, CD267, CD268, CD269, CD355, CD358, CRTH2, NKG2A, NKG2B, NKG2C, NKG2D, NKG2E, NKG2F, NKG2H, KIR2DL1, KIR2DL2, KIR2DL3, KIR2DL5A, KIR2DL5B, KIR3DL1, KIR3DL2, KIR3DL3, KIR3DL4, KIR2DS1, KIR2DS2, KIR2DS3, KIR2DS4, KIR2DS5, DAP12, KIR3DS, NKp44, NKp46, TCR, BCR, Integrins, FcβεRI, MHC-I, MHC-II, IL-1R, IL-2Rα, IL-2Rβ, IL-2Rγ, IL-3Rα, CSF2RB, IL-4R, IL-5Rα, CSF2RB, IL-6Rα, gp130, IL-7Rα, IL-9R, IL-10R, IL-12Rβ1, IL-12Rβ2, IL-13Rα1, IL-13Rα2, IL-15Rα, IL-21R, IL-23R, IL-27Rα, IL-31Rα, OSMR, CSF-1R, cell-surface IL-15, IL-10Rα, IL-10Rβ, IL-20Rα, IL-20Rβ, IL-22Rα1, IL-22Rα2, IL-22Rβ, IL-28RA, PD-1, PD-1H, BTLA, CTLA-4, PD-L1, PD-L2, 2B4, B7-1, B7-2, B7-H1, B7-H4, B7-DC, DR3, LIGHT, LAIR, LTα1β2, LTβR, TIM-1, TIM-3, TIM-4, TIGIT, LAG-3, ICOS, ICOS-L, SLAM, SLAMF2, OX-40, OX-40L, GITR, GITRL, TL1A, HVEM, 41-BB, 41BB-L, TL-1A, TRAF1, TRAF2, TRAF3, TRAF5, BAFF, BAFF-R, APRIL, TRAIL, RANK, AITR, TRAMP, CCR1, CCR2, CCR3, CCR4, CCR5, CCR6, CCR7, CCR8, CCR9, CCR10, CCR11,CXCR1, CXCR2, CXCR3, CXCR4, CXCR5, CXCR6, CXCR7, CLECL9a, DC-SIGN, IGSF4A, SIGLEC, EGFR, PDGFR, VEGFR, FAP, α-SMA, FAS, FAS-L, FC, ICAM-1, ICAM-2, ICAM-3, ICAM-4, ICAM-5, PECAM-1, MICA, MICB, UL16, ULBP1, ULBP2, ILBP3, ULBP4, ULBP5, ULBP6, MULT1, RAE1α,β,γ,δ, and ε, H60a, H60b, H60c, GPR15, ST2, and/or combinations thereof. In various embodiments, Integrins are selected from the group consisting of α1, α2, αIIb, α3, α4, α5, α6, α7, α8, α9, α10, α11, αD, αE, αL, αM, αV, αX, β1, β2, β3, β4, β5, β6, β7, β8 and/or combinations thereof. In various embodiments, TCR is selected from the group consisting of α, β, γ, δ, ε, ζ and/or combinations thereof. Several methods have been described in the literature for assaying of cell-surface protein expression, including Flow Cytometry and Mass Cytometry (CyTOF).

In various embodiments, the subject's tolerance status is determined by analyzing nucleic acids from the biological sample(s). In various embodiments, the nucleic acids are DNA and/or RNA. In various embodiments, the nucleic acids are mRNA, rRNA, tRNA, siRNA, miRNA, lncRNA, and ncRNA and mitochondrial DNA. In various embodiments, the subject's immune tolerance status is determined by assaying gene expression from the biological sample(s). In various embodiments, the immune tolerance status is determined by assaying gene expression associated with immune function, an antibody, foreign body (e.g., bacteria, virus, infections, or natural or synthetic implants) response, metabolism, apoptosis, cell death, necrosis, ferroptosis, autophagy, cell migration, endocytosis, phagocytosis, pinocytosis, tight-junction regulation, cell adhesion, differentiation, and/or combinations thereof. In various embodiments, the immune tolerance status is determined by assaying gene expression associated with immune suppression. In various embodiments, the immune tolerance status is determined by assaying gene expression associated with immune activation. In various embodiments, the immune tolerance status is determined by assaying gene expression associated with immune regulatory functions. In various embodiments, nucleic acid analysis is used to generate an immune tolerance signature. Several methodologies have been described in the literature for high-throughput gene expression analysis including RNA sequencing (RNA-seq), single-cell RNA sequencing (scRNA-seq), exome sequencing, and microarray-based analyses.

In various embodiments, the subject's immune tolerance status is determined by analyzing proteins in the biological sample(s). In various embodiments, the proteins are associated with an immune response, foreign body response, metabolism, apoptosis, cell death, necrosis, ferroptosis, autophagy, cell migration, endocytosis, phagocytosis, DNA damage, pinocytosis, tight-junction regulation, cell adhesion, differentiation, presence and/or absence of cell types, and/or combinations thereof. In various embodiments, the proteins are cytokines and/or chemokines. In various embodiments the proteins are cell signaling proteins. In various embodiments, the cytokines and chemokines are selected from the group consisting of IL-1α, IL-1β, IL-2, IL-3, IL-4, IL-5, IL-6, IL-7, IL-8, IL-9, IL-10, IL-11, IL-12, IL-12p70, IL-13, IL-14, IL-15, IL-16, IL-17, IL-17, IL-18, IL-20, IL-21, IL-22, IL-23, IL-24, IL-25, IL-26, IL-27, IL-27b, IL-28, IL-29, IL-30, IL-31, IL-32, IL-33, IL-35, IL-36, CCL1, CCL2, CCL3, CCL4, CCL5, CCL6, CCL7, CCL8, CCL9, CCL10, CCL11, CCL12, CCL14, CCL15, CCL16, CCL17, CCL18, CCL19, CCL20, CCL21, CCL22, CCL23, CCL24, CCL25, CCL26, CCL27, CCL28, CXCL1, CXCL2 (MCP-1), CXCL3 (MIP-1a, CXCL4 (MIP-1B, CXCL5 (RANTES), CXCL6, CXCL7, CXCL8, CXCL9, CXCL10, CXCL11, CXCL12, CXCL13, CXCL14, CXCL15, CXCL16, CXCL17, GM-CSF, IFN-α, IFN-β, IFN-γ, TNF-α, TGF-β1, TGF-β2, TGF-β3, and/or combinations thereof. In various embodiments, the protein is a protease. In various embodiments, the protease is an aspartic protease, a cysteine protease, a metalloprotease, a serine protease, and/or a threonine protease. In various embodiments, the protease is selected from the group consisting of ADAM1, ADAM2, ADAM7, ADAM8, ADAM9, ADAM10, ADAM11, ADAM12, ADAM15, ADAM17, ADAM18, ADAM19, ADAAM20, ADAM21, ADAM22, ADAM23, ADAM28, ADAM29, ADAM30, ADAM33, MMP1, MMP2, MMP3, MMP7, MMP8, MMP9, MMP10, MMP11, MMP12, MMP13, MMP14, MMP15, MMP16, MMP17, MMP18, MMP19, MMP20, MMP21, MMP23A, MMP23B, MMP24, MMP25, MMP26, MMP27, and MMP28. In various embodiments, proteins associated with apoptosis are selected from the group consisting of P53, Caspase 1, Caspase 2, Caspase 3, Caspase 4, Caspase 5, Caspase 6, Caspase 7, Caspase 8, Caspase 9, Caspase 10, Caspase 11, Caspase 12, Caspase 13, Caspase 14, BCL-2, BCL-XL, MCL-1, CED-9, A1, BFL1, BAX, BAK, DIVA, BCL-XS, BIK, BIM, BAD, BID, and EGL-1. Several methods for assaying proteins from a biological sample have been described in the literature including enzyme-linked immunosorbent assay (ELISA), western blots, and mass spectrometry. In various embodiments the protein is one or more immunoglobulins (Ig). In various embodiments, the Ig are selected from the group consisting of IgA, IgD, IgE, IgM, and/or variants thereof. In various embodiments the immunoglobulins are antigen specific. Several methods for the detection of immunoglobulins from a biological sample have been described in the literature including ELISA and ImmunoCap.

In various embodiments, the immune tolerance signature indicates immune activation, an effector immune response, an effector memory response, a cytotoxic response, immune downregulation, immune suppression, a regulatory immune response, a suppressive response, a TH1 response, a TH2 response, an antibody response, and/or combinations thereof.

In various embodiments, the subject's immune tolerance status is determined by assay of one or more biological samples at baseline. In various embodiments, the baseline is defined from the assay of one or more biological samples collected prior to or after administration of the immune tolerizing therapy. In various embodiments, the baseline is defined from the assay of one or more biological samples collected 1-7 days, 1-4 weeks, and/or 1-12 month prior to or after the administration of the immune tolerizing therapy. In various embodiments, the subject's immune tolerance status is determined by assay of one or more biological samples in response to one or more stimuli. In various embodiments, the stimuli are provided in vivo. In various embodiments, the in vivo stimuli are one or more antigens. In various embodiments, the antigen stimuli comprise ingestion of one or more antigens, intradermal injection of one or more antigens, or intranasal administration of one or more antigens. In various embodiments, the antigen is associated with the disease or condition being treated. In various embodiments, the antigen is not associated with the disease or condition being treated. In various embodiments, the one or more stimuli are ex vivo. In various embodiments, the ex vivo stimuli are provided by incubation of one or more biological samples from the subject with one or more antigens, or incubation with one or more activating agents. In various embodiments, the one or more ex vivo stimuli are provided by immune activating agents consisting of an antibody, a chemical, a bacterial, and/or a viral component. In various embodiments, the immune activating agent comprises a toll-like receptor (TLR) agonist. In various embodiments, the immune activating agent comprises a STING agonist. In various embodiments, the immune activating agent is a chemical agent (e.g., ionomycin, phorbol myristate acetate (PMA), or a calcium channel activator). In various embodiments, the immune activating agent is a T cell activating agent. In various embodiments, the immune activating agent is selected from the group consisting of anti-CD3, anti-CD28, CD40L, ionomycin, phorbol myristate acetate (PMA), or lipopolysaccharide (LPS).

In various embodiments, the method comprises the steps of (a) obtaining one or more biological samples from a subject prior to administration of immune tolerizing therapy and assaying cell-surface protein expression in the biological sample(s) collected, (b) obtaining one or more biological samples from the subject after administration of the immune tolerizing therapy and assaying cell-surface protein expression in the biological sample(s) collected, (c) obtaining one or more biological samples from the subject at regular intervals after administration of the tolerizing therapy and assaying cell-surface protein expression in the biological sample(s) collected, and (d) re-administering the tolerizing therapy if the cell-surface protein expression determined in step (c) indicates a change, weakening, and/or loss of immunological tolerance. In various embodiments, the cell-surface protein expression from Step (c) is compared to the results from Steps (a) and/or (b). In various embodiments, the comparison of cell-surface protein expression from Step (c) to the results from Steps (a) and/or (b) indicates weakening and/or loss of immune tolerance by about 5%-100% (e.g., about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, inclusive of all values and ranges between these values), 10-95%, 15-90%, 20-85%, 25-75%, 30-70%, 35-65%, 40-60%, 45-55%, or 50%. In various embodiments, the comparison of cell-surface protein expression from Step (c) to the results from Steps (a) and/or (b) indicates weakening and/or loss of immune tolerance by about 2-100-fold (e.g., about 2, 5, 10, 15, 20, 25, 30, 35, 40, 45, 50, 55, 60, 65, 70, 75, 80, 85, 90, 95, or 100-fold, inclusive of all values and ranges between these values). In various embodiments, the subject is re-administered the immune tolerizing therapy if the comparison of cell-surface protein expression from the assay of one or more biological samples from Step (c) to the cell-surface protein expression from Steps (a) and/or (b) indicates weakening and/or loss of immune tolerance by about >5% (e.g., about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, inclusive of all values and ranges between these values). In various embodiments, the subject is re-administered the immune tolerizing therapy if the comparison of cell-surface protein expression from the assay of one or more biological samples in Step (c) to the cell-surface protein expression from Steps (a) and/or (b) indicates weakening and/or loss of immune tolerance by about >2-fold (e.g., about 2, 5, 10, 15, 20, 25, 30, 35, 40, 45, 50, 55, 60, 65, 70, 75, 80, 85, 90, 95, or 100-fold, inclusive of all values and ranges between these values).

In various embodiments, the method comprises the steps of (a) obtaining one or more biological samples from a subject prior to administration of immune tolerizing therapy and assaying chemokine and/or cytokine levels in the biological sample(s) collected, (b) obtaining one or more biological samples from the subject after administration of the immune tolerizing therapy and assaying chemokine and/or cytokine levels in the biological sample(s) collected, (c) collecting one or more biological samples from the subject at regular intervals after administration of the tolerizing therapy and assaying chemokine and/or cytokine levels in the biological sample(s) collected, and (d) re-administering the tolerizing therapy if the chemokine and/or cytokine levels determined in step (c) indicate a change, weakening, and/or loss of immunological tolerance. In various embodiments, the comparison of cytokine/chemokine levels from the assay of one or more biological samples from Step (c) to the cytokine/chemokine levels from Steps (a) and/or (b) indicates weakening and/or loss of immune tolerance by about 5%-100% (e.g., about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, inclusive of all values and ranges between these values), 10-95%, 15-90%, 20-85%, 25-75%, 30-70%, 35-65%, 40-60%, 45-55%, or 50%. In various embodiments, the comparison of cytokine/chemokine levels from the assay of one or more biological samples in Step (c) to the cytokine/chemokine levels from Steps (a) and/or (b) indicates weakening and/or loss of immune tolerance by about 2-100-fold (e.g., about 2, 5, 10, 15, 20, 25, 30, 35, 40, 45, 50, 55, 60, 65, 70, 75, 80, 85, 90, 95, or 100-fold, inclusive of all values and ranges between these values). In various embodiments, the subject is re-administered the immune tolerizing therapy in Step (d) if the comparison of cytokine/chemokine levels from the assay of one or more biological samples from Step (c) to the cytokine/chemokine levels from Steps (a) and/or (b) indicates weakening and/or loss of immune tolerance by about >5% (e.g., about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, inclusive of all values and ranges between these values). In various embodiments, the subject is re-administered the immune tolerizing therapy in Step (d) if the comparison of cytokine/chemokine levels from the assay of one or more biological samples in Step (c) to the cytokine/chemokine levels from Steps (a) and/or (b) indicates weakening and/or loss of immune tolerance by about >2-fold (e.g., about 2, 5, 10, 15, 20, 25, 30, 35, 40, 45, 50, 55, 60, 65, 70, 75, 80, 85, 90, 95, or 100-fold, inclusive of all values and ranges between these values).

In various embodiments, the method comprises the steps of (a) obtaining one or more biological samples from a subject prior to administration of immune tolerizing therapy and assaying gene expression pattern in the biological sample(s) collected, (b) obtaining one or more biological samples from the subject after administration of the tolerizing therapy and assaying gene expression pattern in the biological sample(s) collected, (c) collecting one or more biological samples from the subject at regular intervals after administration of the tolerizing therapy and assaying gene expression pattern in the biological sample(s) collected, and (d) re-administering the tolerizing therapy if the gene expression pattern determined in step (c) indicates a change, weakening, and/or loss of immunological tolerance. In various embodiments, the comparison of gene expression from the assay of one or more biological samples from Step (c) to the gene expression from Steps (a) and/or (b) indicates weakening and/or loss of immune tolerance by about 5%-100% (e.g., about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, inclusive of all values and ranges between these values), 10-95%, 15-90%, 20-85%, 25-75%, 30-70%, 35-65%, 40-60%, 45-55%, or 50%. In various embodiments, the comparison of gene expression from the assay of one or more biological samples in Step (c) to the gene expression from Steps (a) and/or (b) indicates weakening and/or loss of immune tolerance by about 2-100-fold (e.g., about 2, 5, 10, 15, 20, 25, 30, 35, 40, 45, 50, 55, 60, 65, 70, 75, 80, 85, 90, 95, or 100-fold, inclusive of all values and ranges between these values). In various embodiments, the subject is re-administered the immune tolerizing therapy in if the comparison of gene expression from the assay of one or more biological samples from Step (c) to the gene expression from Steps (a) and/or (b) indicates weakening and/or loss of immune tolerance by about >5% (e.g., about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, inclusive of all values and ranges between these values). In various embodiments, the subject is re-administered the immune tolerizing therapy if the comparison of gene expression from the assay of one or more biological samples in Step (c) to the gene expression from Steps (a) and/or (b) indicates weakening and/or loss of immune tolerance by about >2-fold (e.g., about 2, 5, 10, 15, 20, 25, 30, 35, 40, 45, 50, 55, 60, 65, 70, 75, 80, 85, 90, 95, or 100-fold, inclusive of all values and ranges between these values).

In various embodiments, the method comprises the steps of (a) obtaining one or more biological samples from a subject prior to administration of immune tolerizing therapy and assaying the levels of intracellular proteins in the biological sample(s) collected, (b) obtaining one or more biological samples from the subject after administration of the immune tolerizing therapy and assaying levels of intracellular proteins in the biological sample(s) collected, (c) collecting one or more biological samples from the subject at regular intervals after administration of the immune tolerizing therapy and assaying levels of intracellular proteins in the biological sample(s) collected, and (d) re-administering the immune tolerizing therapy if the levels of intracellular proteins determined in step (c) indicate a change, weakening, and/or loss of immunological tolerance. In various embodiments, the comparison of levels of intracellular proteins from the assay of one or more biological samples from Step (c) to the levels of intracellular proteins from Steps (a) and/or (b) indicates weakening and/or loss of immune tolerance by about 5%-100% (e.g., about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, inclusive of all values and ranges between these values), 10-95%, 15-90%, 20-85%, 25-75%, 30-70%, 35-65%, 40-60%, 45-55%, or 50%. In various embodiments, the comparison of levels of intracellular proteins from the assay of one or more biological samples in Step (c) to the levels of intracellular proteins from Steps (a) and/or (b) indicates weakening and/or loss of immune tolerance by about 2-100-fold (e.g., about 2, 5, 10, 15, 20, 25, 30, 35, 40, 45, 50, 55, 60, 65, 70, 75, 80, 85, 90, 95, or 100-fold, inclusive of all values and ranges between these values). In various embodiments, the subject is re-administered the immune tolerizing therapy in Step (d) if the comparison of levels of intracellular proteins from the assay of one or more biological samples from Step (c) to the levels of intracellular proteins from Steps (a) and/or (b) indicates weakening and/or loss of immune tolerance by about >5% (e.g., about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, inclusive of all values and ranges between these values). In various embodiments, the subject is re-administered the immune tolerizing therapy if the comparison of levels of intracellular proteins from the assay of one or more biological samples in Step (c) to the levels of intracellular proteins from Steps (a) and/or (b) indicates weakening and/or loss of immune tolerance by about >2-fold (e.g., about 2, 5, 10, 15, 20, 25, 30, 35, 40, 45, 50, 55, 60, 65, 70, 75, 80, 85, 90, 95, or 100-fold, inclusive of all values and ranges between these values).

In various embodiments, the method comprises the steps of (a) obtaining one or more biological samples from a subject prior to administration of immune tolerizing therapy and assaying the levels of extracellular proteins in the biological sample(s) collected, (b) obtaining one or more biological samples from the subject after administration of the immune tolerizing therapy and assaying levels of extracellular proteins in the biological sample(s) collected, (c) collecting one or more biological samples from the subject at regular intervals after administration of the immune tolerizing therapy and assaying levels of extracellular proteins in the biological sample(s) collected, and (d) re-administering the immune tolerizing therapy if the levels of extracellular proteins determined in step (c) indicate a change, weakening, and/or loss of immunological tolerance. In various embodiments, the comparison of levels of extracellular proteins from the assay of one or more biological samples from Step (c) to the levels of extracellular proteins from Steps (a) and/or (b) indicates weakening and/or loss of immune tolerance by about 5%-100% (e.g about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, inclusive of all values and ranges between these values), 10-95%, 15-90%, 20-85%, 25-75%, 30-70%, 35-65%, 40-60%, 45-55%, or 50%. In various embodiments, the comparison of levels of extracellular proteins from the assay of one or more biological samples in Step (c) to the levels of extracellular proteins from Steps (a) and/or (b) indicates weakening and/or loss of immune tolerance by about 2-100-fold (e.g., about 2, 5, 10, 15, 20, 25, 30, 35, 40, 45, 50, 55, 60, 65, 70, 75, 80, 85, 90, 95, or 100-fold, inclusive of all values and ranges between these values). In various embodiments, the subject is re-administered the immune tolerizing therapy in Step (d) if the comparison of levels of extracellular proteins from the assay of one or more biological samples from Step (c) to the levels of extracellular proteins from Steps (a) and/or (b) indicates weakening and/or loss of immune tolerance by about >5% (e.g., about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, inclusive of all values and ranges between these values). In various embodiments, the subject is re-administered the immune tolerizing therapy if the comparison of levels of extracellular proteins from the assay of one or more biological samples in Step (c) to the levels of extracellular proteins from Steps (a) and/or (b) indicates weakening and/or loss of immune tolerance by about >2-fold (e.g., about 2, 5, 10, 15, 20, 25, 30, 35, 40, 45, 50, 55, 60, 65, 70, 75, 80, 85, 90, 95, or 100-fold, inclusive of all values and ranges between these values).

In various embodiments, the method comprises the steps of (a) obtaining one or more biological samples from a subject prior to administration of immune tolerizing therapy and assaying the levels of metabolites in the biological sample(s) collected, (b) obtaining one or more biological samples from the subject after administration of the immune tolerizing therapy and assaying levels of metabolites in the biological sample(s) collected, (c) collecting one or more biological samples from the subject at regular intervals after administration of the immune tolerizing therapy and assaying levels of metabolites in the biological sample(s) collected, and (d) re-administering the tolerizing therapy if the levels of metabolites determined in step (c) indicate a change, weakening, and/or loss of immunological tolerance. In various embodiments, the comparison of levels of extracellular proteins from the assay of one or more biological samples from Step (c) to the levels of metabolites from Steps (a) and/or (b) indicates weakening and/or loss of immune tolerance by about 5%-100% (e.g., about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, inclusive of all values and ranges between these values), 10-95%, 15-90%, 20-85%, 25-75%, 30-70%, 35-65%, 40-60%, 45-55%, or 50%. In various embodiments, the comparison of levels of metabolites from the assay of one or more biological samples in Step (c) to the levels of metabolites from Steps (a) and/or (b) indicates weakening and/or loss of immune tolerance by about 2-100-fold (e.g., about 2, 5, 10, 15, 20, 25, 30, 35, 40, 45, 50, 55, 60, 65, 70, 75, 80, 85, 90, 95, or 100-fold, inclusive of all values and ranges between these values). In various embodiments, the subject is re-administered the immune tolerizing therapy in Step (d) if the comparison of levels of metabolites from the assay of one or more biological samples from Step (c) to the levels of metabolites from Steps (a) and/or (b) indicates weakening and/or loss of immune tolerance by about >5% (e.g., about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, inclusive of all values and ranges between these values). In various embodiments, the subject is re-administered the immune tolerizing therapy if the comparison of levels of metabolites from the assay of one or more biological samples in Step (c) to the levels of metabolites from Steps (a) and/or (b) indicates weakening and/or loss of immune tolerance by about >2-fold (e.g., about 2, 5, 10, 15, 20, 25, 30, 35, 40, 45, 50, 55, 60, 65, 70, 75, 80, 85, 90, 95, or 100-fold, inclusive of all values and ranges between these values).

In various embodiments, the immune tolerance signature of a subject is generated using one or more of the following parameters assayed from one or more biological samples obtained from the subject and stimulated in vivo and/or ex vivo:
a. proportion of effector T cells in the total T cell population,
b. proportion of Treg cells in the total T cell population,
C. proportion of effector B cells in the total B cell population,
d. levels and/or ratios of specific IgG, IgA, IgM, and/or IgE,
e. levels of inflammatory cytokines and chemokines,
f. levels of anti-inflammatory cytokines and chemokines,
g. levels of inflammatory metabolites, and
h. levels of anti-inflammatory metabolites.

In various embodiments, the biological sample is optionally assayed after in vivo and/or ex vivo stimulation with one or more stimuli selected from the group consisting of an antigen, an allergen, and one or more activating agents. In various embodiments, the T cells, B cells, and immunoglobulins are antigen specific. In various embodiments, the T cells are effector memory T cells, antigen specific T cells, activated antigen specific T cells, Th1 cells, pathogenic Th2a+ cells, Th17 cells, T follicular helper (TFH) cells, or Th0 cells. In various embodiments, the B cells are effector B cells, memory B cells, plasma B cells, and regulatory (Breg) cells. In various embodiments, T cells are identified based on the expression of proteins described in Table A (see Detailed Description).

In various embodiments, the immune tolerance signature of a subject generated using one or more parameters described herein indicates weakening and/or absence of immune tolerance prior to or after treatment with TIMPs if:
a. the proportion of effector T cells in the total T cell population is between 5%-100% (e.g., about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, inclusive of all values and ranges between these values), and/or
b. the proportion of Treg cells in the total T cell population is between 1-3%, and/or
c. the proportion of effector B cells in the total B cell population is between 5%-100% (e.g., about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, inclusive of all values and ranges between these values), and/or
d. the levels and/or ratios of IgG, IgA, IgM, and/or IgE are increased by about 5%-100% (e.g. about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, inclusive of all values and ranges between these values), 10-95%, 15-90%, 20-85%, 25-75%, 30-70%, 35-65%, 40-60%, 45-55%, or 50% or by about 2-100-fold (e.g., about 2, 5, 10, 15, 20, 25, 30, 35, 40, 45, 50, 55, 60, 65, 70, 75, 80, 85, 90, 95, or 100-fold inclusive of all values and ranges between these values) relative to a healthy subject and/or one or more baseline measurements taken from the subject during treatment, and/or
e. levels of inflammatory cytokines/chemokines are increased by about 5%-100% (e.g. about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, inclusive of all values and ranges between these values), 10-95%, 15-90%, 20-85%, 25-75%, 30-70%, 35-65%, 40-60%, 45-55%, or 50% or by about 2-100-fold (e.g., about 2, 5, 10, 15, 20, 25, 30, 35, 40, 45, 50, 55, 60, 65, 70, 75, 80, 85, 90, 95, or 100-fold inclusive of all values and ranges between these values) relative to a healthy subject and/or one or more baseline measurements taken from the subject during treatment, and/or
f. levels of anti-inflammatory cytokines and chemokines are decreased by about 5%-100% (e.g. about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, inclusive of all values and ranges between these values), 10-95%, 15-90%, 20-85%, 25-75%, 30-70%, 35-65%, 40-60%, 45-55%, or 50% or by about 2-100-fold (e.g., about 2, 5, 10, 15, 20, 25, 30, 35, 40, 45, 50, 55, 60, 65, 70, 75, 80, 85, 90, 95, or 100-fold inclusive of all values and ranges between these values) relative to a healthy subject and/or one or more baseline measurements taken from the subject during treatment, and/or
g. levels of inflammatory metabolites are increased by about 5%-100% (e.g. about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, inclusive of all values and ranges between these values), 10-95%, 15-90%, 20-85%, 25-75%, 30-70%, 35-65%, 40-60%, 45-55%, or 50% or by about 2-100-fold (e.g., about 2, 5, 10, 15, 20, 25, 30, 35, 40, 45, 50, 55, 60, 65, 70, 75, 80, 85, 90, 95, or 100-fold inclusive of all values and ranges between these values) relative to a healthy subject and/or one or more baseline measurements taken from the subject during treatment, and/or
h. levels of anti-inflammatory metabolites are decreased by about 5%-100% (e.g. about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, inclusive of all values and ranges between these values), 10-95%, 15-90%, 20-85%, 25-75%, 30-70%, 35-65%, 40-60%, 45-55%, or 50% or by about 2-100-fold (e.g., about 2, 5, 10, 15, 20, 25, 30, 35, 40, 45, 50, 55, 60, 65, 70, 75, 80, 85, 90, 95, or 100-fold inclusive of all values and ranges between these values) relative to a healthy subject and/or one or more baseline measurements taken from the subject during treatment.

In various embodiments, the immune tolerance signature is indicative of weakening and/or absence of immune tolerance if any 1, 2, 3, 4, 5, 6, 7, or 8 parameters listed in (a)-(h) above indicate weakening and/or loss of immune tolerance. In various embodiments, the immune tolerance signature is indicative of weakening and/or absence of immune tolerance if at least 2/8 parameters listed (a)-(h) above indicate weakening and/or loss of immune tolerance. In various embodiments, the subject is administered TIMPs if 1, 2, 3, 4, 5, 6, 7, or 8 parameters listed (a)-(h) above are determined to indicate weakening and/or absence of immune tolerance. In various embodiments, the subject is administered TIMPs if at least 2/8 parameters listed in (a)-(h) above are determined to indicate weakening and/or absence of immune tolerance.

In various embodiments, the immune tolerance signature of a subject generated using one or more parameters described in (a)-(h) above indicates maintenance of immune tolerance after treatment with TIMPs if:
a. the proportion of effector T cells in the total T cell population is <5%;
b. the proportion of Treg cells in the total T cell population is at least 3% or >3%;
c. the proportion of effector B cells in the total B cell population is <5%;
d. the levels and/or ratios of IgG, IgA, IgM, and/or IgE are decreased by about 5%-100% (e.g. about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, inclusive of all values and ranges between these values), 10-95%, 15-90%, 20-85%, 25-75%, 30-70%, 35-65%, 40-60%, 45-55%, or 50% or by about 2-100-fold (e.g., about 2, 5, 10, 15, 20, 25, 30, 35, 40, 45, 50, 55, 60, 65, 70, 75, 80, 85, 90, 95, or 100-fold inclusive of all values and ranges between these values) relative to the Baseline determined from one or more biological samples collected prior to treatment with TIMPs;
e. the levels of inflammatory cytokines and chemokines are decreased by about 5%-100% (e.g., about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, inclusive of all values and ranges between these values), 10-95%, 15-90%, 20-85%, 25-75%, 30-70%, 35-65%, 40-60%, 45-55%, or 50% or by about 2-100-fold (e.g., about 2, 5, 10, 15, 20, 25, 30, 35, 40, 45, 50, 55, 60, 65, 70, 75, 80, 85, 90, 95, or 100-fold inclusive of all values and ranges between these values) relative to the Baseline determined from one or more biological samples collected prior to treatment with TIMPs;
f. the levels of anti-inflammatory cytokines and chemokines are increased by about 5%-100% (e.g., about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, inclusive of all values and ranges between these values), 10-95%, 15-90%, 20-85%, 25-75%, 30-70%, 35-65%, 40-60%, 45-55%, or 50% or by about 2-100-fold (e.g., about 2, 5, 10, 15, 20, 25, 30, 35, 40, 45, 50, 55, 60, 65, 70, 75, 80, 85, 90, 95, or 100-fold inclusive of all values and ranges between these values) relative to the Baseline determined from one or more biological samples collected prior to treatment with TIMPs;
g. levels of inflammatory metabolites are decreased by about 5%-100% (e.g about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, inclusive of all values and ranges between these values), 10-95%, 15-90%, 20-85%, 25-75%, 30-70%, 35-65%, 40-60%, 45-55%, or 50% or by about 2-100-fold (e.g., about 2, 5, 10, 15, 20, 25, 30, 35, 40, 45, 50, 55, 60, 65, 70, 75, 80, 85, 90, 95, or 100-fold inclusive of all values and ranges between these values) relative to the Baseline determined from one or more biological samples collected prior to treatment with TIMPs; and/or,
h. levels of anti-inflammatory metabolites are increased by about 5%-100% (e.g about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, inclusive of all values and ranges between these values), 10-95%, 15-90%, 20-85%, 25-75%, 30-70%, 35-65%, 40-60%, 45-55%, or 50% or by about 2-100-fold (e.g., about 2, 5, 10, 15, 20, 25, 30, 35, 40, 45, 50, 55, 60, 65, 70, 75, 80, 85, 90, 95, or 100-fold inclusive of all values and ranges between these values) relative to the Baseline determined from one or more biological samples collected prior to treatment with TIMPs.

In various embodiments, the immune tolerance signature is indicative of maintenance of immune tolerance if 1, 2, 3, 4, 5, 6, 7, or 8 parameters listed in (a)-(h) above indicate maintenance of immune tolerance. In various embodiments, the immune tolerance signature is indicative of maintenance of immune tolerance if at least 2/8 parameters listed in (a)-(h) indicate maintenance of immune tolerance. In various embodiments, the subject is determined to not require treatment with TIMPs if 1, 2, 3, 4, 5, 6, 7, or 8 parameters listed in (a)-(h) above indicate maintenance of immune tolerance. In various embodiments, the subject is determined to not require treatment with TIMPs if at least 3/8 parameters listed in (a)-(h) above indicate maintenance of immune tolerance.

In various embodiments, the subject maintains immune tolerance for 1-3 months after administration of the treatment. In various embodiments, the subject maintains immune tolerance for 1-12 weeks. In various embodiments, the subject maintains tolerance for 1, 2, or 3 months. In various embodiments, maintenance of immune tolerance for <3 months is indicative of short-term tolerance requiring re-administration of the treatment. In various embodiments, the subject maintains immune tolerance of 3-6 months after administration of treatment. In various embodiments, the subject maintains immune tolerance for 12-24 weeks. In various embodiments, the subject maintains immune tolerance for 6-12 months. In various embodiments, the subject maintains immune tolerance for 13-52 weeks. In various embodiments, the subject maintains tolerance for >12 months. In various embodiments, maintenance of immune tolerance for >12 months indicates long-term tolerance.

In various embodiments, the subject is suffering from or being treated for a disease or condition. In various embodiments, the subject has or is being treated for an autoimmune condition, an allergy, an inflammatory disease, an abnormal immune response, a hyperinflammatory condition, a neurodegenerative condition, a lysosomal storage disease, an enzyme deficiency, a protein deficiency, a genetic disorder, and/or is a transplant recipient.

In various embodiments, the autoimmune condition is selected from the group consisting of atopic dermatitis, multiple sclerosis, autoimmune myelitis, myelitis, transverse myelitis, neuromyelitis optica (NMO), neuromyelitis optica spectrum disorder (NMSOD), type-1 diabetes (T1D), type-2 diabetes (T2D), Celiac Disease (CD), Grave's Disease, Myasthenia Gravis, acute disseminated encephalomyelitis, Addison's Disease, alopecia, rheumatoid arthritis, osteoarthritis, ankylosing spondylitis, autoimmune myocarditis, autoimmune neutropenia, autoimmune skin disease, autoimmune uveitis, ballus pemphigoid, Behcet's Syndrome, cerebral degeneration, chronic neuropathy, cicatrical pemphigoid, pemphigus vulgaris, Crohn's Disease, Inflammatory Bowel Disease (IBD), colitis, Inflammatory Bowel Syndrome (IBS), cryopathy, dermatitis hyperformis, Eaton Lambert's Disease, encephalomyelitis, epidermolysis bullosa acquisita, erythema nodosa, glomerulonephritis, Goodpasture's Disease, granulomatosis, Guillain-Barre Syndrome, Hashimoto Disease, Kawasaki Disease, hemolytic anemia, hypersensitivity vasculitis, lupus erythematosus, mixed connective tissue disease, mixed essential cryoglobulinemia, multifocal motor neuropathy, opsonoclonus-myoclonus, paraneoplastic pemphigus, pemphigoig gestationis, pemphigus folaceus, pernicious anemia, peripheral biliary cirrhosis (PBC), polyangiitis overlap syndrome, polyarteritis nodosa, polyglandular failure, polyglandular syndrome, polymyositis/dermatomyositis, psoriasis, eczema, retinopathy, Reynaud's Syndrome, sarcoidosis, Scleroderma Type 1, sclerosis cholangitis, Sjogren's Syndrome, Stiffman's syndrome, Takayasu arteritis, termporal arteritis, thyroiditis, ulcerative colitis, immune thrombocytopenia purpura (ITP), thrombotic thrombocytopenia purpura (TTP), autoimmune hepatitis (AIH), primary biliary cholangitis (PBC), ANCA diseases, Granulamatosis with Polyangiitis, and Microscopic Polyangiitis.

In various embodiments, the subject has a food allergy and/or an environmental allergy. In various embodiment, the food allergy is selected from the group consisting of peanut allergy, tree-nut allergy, nut allergy, fish allergy, milk allergy, shellfish allergy, celery allergy, peach allergy, meat allergy, soy allergy, and wheat allergy. In various embodiments, the environmental allergy is selected from the group consisting of dust mite allergy, pollen allergy, mold allergy, dander allergy, Japanese Cedar Pollen allergy, dust mite allergy, cat allergy, dog allergy, and bee venom allergy.

In various embodiments, the subject is undergoing treatment for an autoimmune condition, an allergy, an inflammatory disease, an abnormal immune response, a lysosomal storage disease, an enzyme deficiency, a protein deficiency, a genetic disorder, and/or is a transplant recipient. In various embodiments, the subject is undergoing treatment with an antigen-specific immune tolerizing therapy. In various embodiments, the antigen-specific immune tolerizing therapy induces immunological tolerance to an autoimmune antigen, a transplant antigen, an allergen, an enzyme replacement therapy, a protein therapeutic, and/or a gene therapy vector.

In various embodiments, the autoimmune antigen is selected from the group consisting of myelin basic protein, acetylcholine receptor, endogenous antigen, myelin oligodendrocyte glycoprotein (MOG), myelin basic protein (MBP), proteolipid protein (PLP), myelin associated glycoprotein (MAG), cyclic nucleotide phosphohydrolase, pancreatic beta-cell antigen, insulin, proinsulin, islet-specific glucose-6-phophatase catalytic subunit-related protein (IGRP), glutamic acid decarboxylase (GAD), collagen type 11, human cartilage gp39, fp130-RAPS, fibrillarin, small nucleolar protein, thyroid stimulating factor receptor, histones, glycoprotein gp70, pyruvate dehydrogenase dehydrolipoamide acetyltransferase (PCD-E2), hair follicle antigen, aqua porin 4, Desmoglein 1, Desmoglein 3, nicotinic acetylcholine receptor, gliadin, ADAMTS13, GPIIb/GPIIIa, CYP2D6, BP180, NC16, BP230, Ro60, MPO, thyroid stimulating hormone receptor, and human tropomyosin isoform 5.

In various embodiments, the allergen is selected from the group consisting of Bahia grass pollen (BaGP), peach allergen, milk allergens, celery allergens, nut allergens, bovine serum albumin, Hazelnut allergens, ovalbumin, egg allergen, peanut allergens, fish allergens, shellfish allergens, pollen allergen, tree nut allergen, cat allergen, dog allergen, dust mite allergen, and Japanese cedar pollen. In various embodiments, the peanut allergen is selected from the group consisting of Ara h1, Ara h2, Ara h3, Ara h5, Ara h6, Ara h7, and Ara h8. In various embodiments, the peanut allergen is selected from the group consisting of Ara h1, Ara h2, Ara h3, Ara h5, Ara h6, Ara h7, Ara h8, Ara h9, Ara h10, Ara h11, Ara h12, Ara h13, Ara h14, Ara h15, Ara h16, Ara h17 and Ara h18.

In various embodiments, the enzyme replacement therapy is selected from the group consisting of Agalsidase beta, Agalsidase alfa, Imiglucirase, Taliglucirase alfa, Velaglucerase alfa, Alglucerase, Sebelipase alpha, Laronidase, Idursulfase, Elosulfase alpha, Galsulfase, Alglucosidase alpha, Factor VII, Factor VIII, Factor IX, Acetylgalactosamine 4-sulfate, Iduronidase, Alglucerase, Glucocerebrosidase, or versions thereof.

In various embodiments, the protein therapeutic is a recombinant protein. In various embodiments, the protein therapeutic is selected from the group consisting of erythropoietin, insulin, human growth hormone, follicle-stimulating hormone, granulocyte colony-stimulating factor, tissue plasminogen activator, insulin-like growth factor, uricase, kynurinase, L-arginine deaminase, arginase, methionine-γ-lyase, asparaginase, an amino acid degrading enzyme, a gluten degrading enzyme, a nucleotide degrading enzyme, IFN-ß, IL-2, IL-12, and IL-15.

In various embodiments, the protein therapeutic is an antibody. In various embodiments, the antibody is a monoclonal antibody or a polyclonal antibody. In various embodiments, the antibody is mono-specific, bi-specific, tri-specific, or bi-specific T-cell engager. In various embodiments the antibody targets receptor tyrosine kinase (RTK), EGFR, VEGF, VEGFR, PDGF, PDGFR, HER2/Neu, ER, PR, TGF-β1, TGF-β2, TGF-β3, SIRP-α, PD-1, PD-L1, CTLA-4, CD3, CD25, CD19, CD20, CD39, CD47, CD73, FAP, IL-1B, IL-12, IL-2R, IL-15R, IL-23, IL-33, IL-2R, IL-4Rα, T-cells, B-cells, NK cells, macrophages, monocytes, and/or neutrophils. In various embodiments, the antibody is selected from the group consisting of abciximab, adalimumab, alemtuzumab, avelumab, azetolizumab, basiliximab, bevacizumab, bezlotoxumab, blinatumomab, canakinumab, certolizumab, cetuximab, daclizumab, denosumab, durvalumab, efalizumab, emicizumab, etokimab, golimumab, ipilimumab, ixekizumab, infliximab, natalizumab, nivolumab, olaratumab, omalizumab, ofatimumab, palivizumab, panitumumab, pembrolizumab, ramucirumab, rituximab, tocilizumab, trastuzumab, tremelimumab, secukinumab, ustekinumab, and vedolizumab.

In various embodiments the gene therapy vector is a viral or bacterial vector. In various embodiments, the viral vector is selected from the group consisting of adenovirus, adeno-associated virus (AAV), herpes simplex virus, lentivirus, retrovirus, alphavirus, flavivirus, rhabdovirus, measles virus, Newcastle disease virus, poxvirus, vaccinia virus, modified Ankara virus, vesicular stomatitis virus, picornavirus, tobacco mosaic virus, potato virus x, comovirus or cucumber mosaic virus. In various embodiments, the virus is an oncolytic virus. In various embodiments the virus is a chimeric virus, a synthetic virus, a mosaic virus or a pseudotyped virus. In various embodiments, the AAV vector is AAV1, AAV2, AAV3, AAV4, AAV5, AAV6, AAV7, AAV8, AAV9, AAV12, Anc80 or combinations thereof.

### BRIEF DESCRIPTION OF THE FIGURES

FIG. 1. Sample schedule for procedures for determining whether a subject has maintained immune tolerance after treatment with TIMPs and whether re-administration of TIMPs is required for maintenance of immune tolerance.

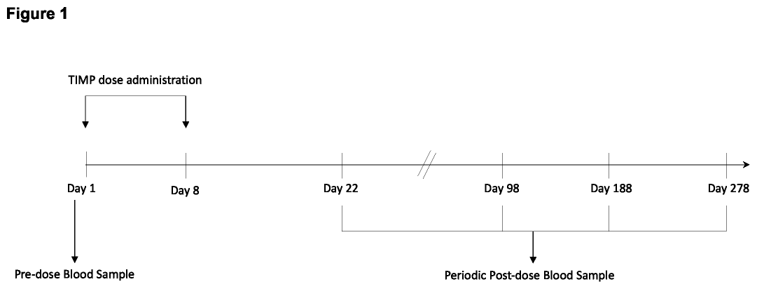

FIG. 2. Sample workflow for determining the immune tolerance status of a subject being treated with TIMPs from one or more blood samples.

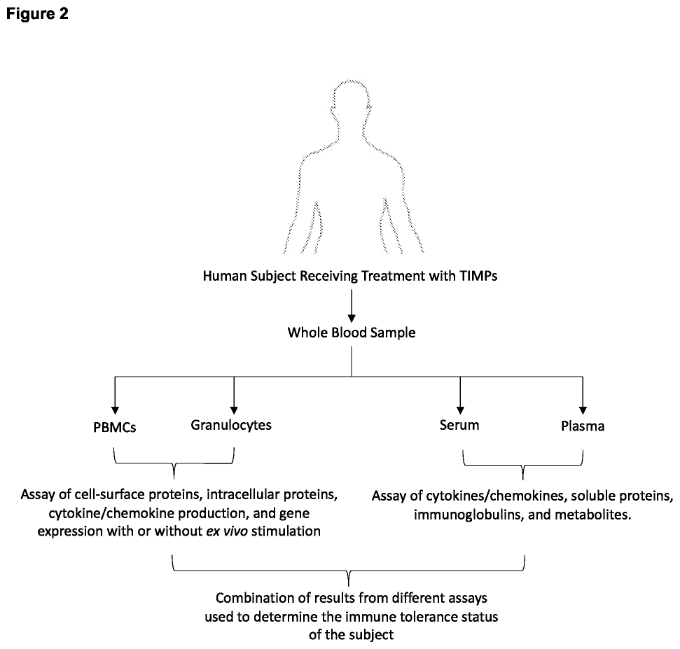

### DETAILED DESCRIPTION

The present disclosure provides methodology for monitoring the induction of and maintenance of immunologic tolerance in a subject after receiving immunotherapy. The present application is the first to disclose a system of assays and readouts of multiple parameters of a subject's immune response before, during and after administration of therapy and provides a method for determining if the subject has maintained antigen specific tolerance or if tolerance has waned and additional tolerizing therapy is needed.

### Definitions

Unless otherwise stated, the following terms used in this application, including the specification and claims, have the definitions given below.

As used in the specification and the appended claims, the indefinite articles “a” and “an” and the definite article “the” include plural as well as singular referents unless the context clearly dictates otherwise.

The term “about” or “approximately” means an acceptable error for a particular value as determined by one of ordinary skill in the art, which depends in part on how the value is measured or determined. In certain embodiments, the term “about” or “approximately” means within 1, 2, 3, or 4 standard deviations. In certain embodiments, the term “about” or “approximately” means within 30%, 25%, 20%, 15%, 10%, 9%, 8%, 7%, 6%, 5%, 4%, 3%, 2%, 1%, 0.5%, or 0.05% of a given value or range. Whenever the term “about” or “approximately” precedes the first numerical value in a series of two or more numerical values, it is understood that the term “about” or “approximately” applies to each one of the numerical values in that series.

“Particle” as used herein refers to any non-tissue derived composition of matter, it may be a sphere or sphere-like entity, bead, or liposome. The term “particle”, the term “immune modifying particle”, the term “carrier particle”, and the term “bead” may be used interchangeably depending on the context. Additionally, the term “particle” may be used to encompass beads and spheres.

“Negatively charged particle” as used herein refers to particles which have been modified to possess a net surface charge that is less than zero.

“Carboxylated particles” or “carboxylated beads” or “carboxylated spheres” includes any particle that has been modified to contain a carboxyl group on its surface. In some embodiments the addition of the carboxyl group enhances phagocyte/monocyte uptake of the particles from circulation, for instance through the interaction with scavenger receptors such as MARCO. Carboxylation of the particles can be achieved using any compound which adds carboxyl groups.

As used herein, the term “Th cell” or “helper T cell” refers to CD4⁺ cells. CD4⁺ T cells assist other white blood cells with immunologic processes, including maturation of B cells into plasma cells and memory B cells, and activation of cytotoxic T cells and macrophages. T cells become activated when they are presented with peptide antigens by MHC class II molecules, which are expressed on the surface of antigen-presenting cells (APCs).

As used herein, the term “Th1 cell” refers to a subset of Th cells which produce proinflammatory mediators. Th1 cells secrete cytokines to facilitate immune response and play a role in host defense against pathogens in part by mediating the recruitment of neutrophils and macrophages to infected tissues. Th1 cells secrete cytokines including IFN-gamma, IL-2, IL-10, and TNF alpha/beta to coordinate defense against intracellular pathogens such as viruses and some bacteria.

As used herein, the term “Th2 cell” refers to a subset of Th cells that mediate the activation and maintenance of the antibody-mediated immune response against extracellular parasites, bacteria, allergens, and toxins. Th2 cells mediate these functions by producing various cytokines such as IL-4, IL-5, IL-6, IL-9, IL-13, and IL-17E (IL-25) that are responsible for antibody production, eosinophil activation, and inhibition of several macrophage functions, thus providing phagocyte-independent protective responses.

“Polypeptide” and “protein” refer to a polymer composed of amino acid residues, related naturally occurring structural variants, and synthetic non-naturally occurring analogs thereof, linked via peptide bonds or peptide bond isosteres. Synthetic polypeptides can be synthesized, for example, using an automated polypeptide synthesizer. The terms “polypeptide” and “protein” are not limited to a minimum length of the product. The term “protein” typically refers to large polypeptides. The term “peptide” typically refers to short polypeptides. Thus, peptides, oligopeptides, dimers, multimers, and the like, are included within the definition. Both full-length proteins and fragments thereof are encompassed by the definition. The terms “polypeptide” and “protein” also include postexpression modifications of the polypeptide or protein, for example, glycosylation, acetylation, phosphorylation and the like. Furthermore, for purposes of the present disclosure, a “polypeptide” can include “modifications,” such as deletions, additions, substitutions (which may be conservative in nature or may include substitutions with any of the 20 amino acids that are commonly present in human proteins, or any other naturally or non-naturally-occurring or atypical amino acids), and chemical modifications (e.g., addition of or substitution with peptidomimetics), to the native sequence. These modifications may be deliberate, as through site-directed mutagenesis, or through chemical modification of amino acids to remove or attach chemical moieties, or may be accidental, such as through mutations arising with hosts that produce the proteins or through errors due to PCR amplification.

“Antigenic moiety” or “antigen” as used herein refers to any moiety, for example a peptide, that is recognized by the host's immune system. Examples of antigenic moieties include, but are not limited to, autoantigens, allergens, enzymes, therapeutic proteins, and/or bacterial or viral proteins, peptides, drugs or components present in drug formulations (e.g., carriers, buffers, and excipients).

“Immune tolerance status” as used herein refers to the level of antigen specific tolerance in a subject either before, during or after receiving a tolerizing therapy. Immune tolerance status can be determined using one or more parameters described herein that are indicative of immune tolerance, e.g., levels of cell surface markers, cytokine profile, cellular proliferation in response to antigen, number and ratio of immune cell populations. “Immune tolerance signature” as used herein refers to the collective pattern of immune tolerance assays which a subject may have before, during or after tolerizing therapy. For example, a subject may have an immune tolerance signature indicating presence or absence of tolerance based on 1, 2, 3, 4, 5, 6, 7 or more tolerance parameters measured. Exemplary tolerance parameters include proportion of effector T cells in the total T cell population, proportion of Treg cells in the total T cell population, proportion of effector B cells in the total B cell population, levels of specific IgG. IgA, IgM, and/or IgE, levels of inflammatory cytokines and chemokines, levels of anti-inflammatory cytokines and chemokines, levels of inflammatory metabolites, and levels of anti-inflammatory metabolites.

“Weakening and/or loss of immune tolerance” as used herein refers to a change in tolerance parameters as measured in the subject which are indicative of a loss of immune tolerance in the subject. Such parameters include an increase in antigen specific T cells, a decrease in the proportion of Treg cells in the total T cell population, a decrease in the proportion of effector B cells in the total B cell population, an increase in IgE levels compared to levels of specific IgG, IgA, IgM, an increase in levels of inflammatory cytokines and chemokines, a decrease in levels of anti-inflammatory cytokines and chemokines, an increase in levels of inflammatory metabolites, and a decrease in levels of anti-inflammatory metabolites. For example, parameters that indicate weakening or loss of tolerance include, but are not limited to, an increase in the frequency of CD4+T effector cells, a decrease in the frequency of antigen specific Treg cells, an increase in the levels of antigen specific antibodies, an increase in IFN-γ production from PBMCs, and a decrease in the ratio of IL-5 to IFN-γ following cell activation in vitro.

“Levels of anti-inflammatory metabolites” refers to intermediates or end products of metabolism which are associated with suppression or downregulation of inflammatory immune responses. Examples of metabolites which are associated with suppression and/or negative regulation of inflammatory immune responses include major classes of metabolites, but are not limited to, acids, lipids, sugars, and amino acids. Examples of such metabolites include, but are not limited to, kynurenine, 3-hydroxy kynurenine, 2-amino-3-carboxymuconic 6-semialdehyde, picolinic acid, anthranilic acid, 3-hydroxylanthranilic acid, glutaryl co-A, NAD+, quinolinic acid, arginine, butyrate, and adenosine. A list of human metabolites that can be assayed from a biological sample can be found in the literature including in (Psychogios et al., 2011), (Wishart et al., HMDB: the Human Metabolome Database. Nucleic Acids Res. 2007 January; 35 (Database issue): D521-6, 2007), and the Human Metabalome Database (HMDB) and are incorporated herein by reference.

“Levels of inflammatory metabolites” refers to intermediates or end products of metabolism which are associated with the induction and/or upregulation of inflammatory immune responses. Examples of metabolites which are associated with induction or upregulation of inflammatory immune responses include major classes of metabolites, but are not limited to, acids, lipids, sugars, and amino acids. Examples include, but are not limited to, lactate, trimethylamine N-oxide, O-acetyl carnitine, L-carnitine, choline, succinate, glutamine, fatty acids, cholesterol, 3-hydroxybutyrate, 3′-sialyllactose, arachidonic acid, prostaglandin (G2 and H2), PGD2, PGE2, PGF2a, PGI2, TXA2, leukotrienes (A4, B4, C4, D4, E4), lipoxin A4, and lipoxin B4.

“Pharmaceutically acceptable carrier” refers to any of the standard pharmaceutical carriers, buffers, and the like, such as a phosphate buffered saline solution, 5% aqueous solution of dextrose, and emulsions (e.g., an oil/water or water/oil emulsion). Non-limiting examples of excipients include adjuvants, binders, fillers, diluents, disintegrants, emulsifying agents, wetting agents, lubricants, glidants, sweetening agents, flavoring agents, and coloring agents. Suitable pharmaceutical carriers, excipients and diluents are described in Remington's Pharmaceutical Sciences, 19th Ed. (Mack Publishing Co., Easton, 1995). Preferred pharmaceutical carriers depend upon the intended mode of administration of the active agent. Typical modes of administration include enteral (e.g., oral) or parenteral (e.g., subcutaneous, intramuscular, intravenous or intraperitoneal injection; or topical, transdermal, or transmucosal administration).

By “pharmaceutically acceptable” or “pharmacologically acceptable” is meant a material that is not biologically or otherwise undesirable, i.e., the material may be administered to an individual without causing any undesirable biological effects or without interacting in a deleterious manner with any of the components of the composition in which it is contained or with any components present on or in the body of the individual.

As used herein, the term “subject” encompasses mammals and non-mammals. Examples of mammals include, but are not limited to, any member of the mammalian class: humans, non-human primates such as chimpanzees, and other apes and monkey species; farm animals such as cattle, horses, sheep, goats, swine; domestic animals such as rabbits, dogs, and cats; laboratory animals including rodents, such as rats, mice and guinea pigs, and the like. Examples of non-mammals include, but are not limited to, birds, fish, and the like. The term does not denote a particular age or gender.

The term “epitope” refers to that portion of any molecule capable of being recognized by and bound by a selective binding agent at one or more of the antigen binding regions. Epitopes usually consist of chemically active surface groupings of molecules, such as, amino acids or carbohydrate side chains, and have specific three-dimensional structural characteristics as well as specific charge characteristics. Epitopes as used herein may be contiguous or non-contiguous. Moreover, epitopes may be mimetic (mimotopes) in that they comprise a three dimensional structure that is identical to the epitope used to generate the antibody, yet comprise none or only some of the amino acid residues found in the target that were used to stimulate the antibody immune response. As used herein, a mimotope is not considered a different antigen from the epitope bound by the selective binding agent; the selective binding agent recognizes the same three-dimensional structure of the epitope and mimotope.

The term “therapeutically effective amount” is used herein to indicate the amount of antigen-specific composition of the disclosure that is effective to ameliorate or lessen symptoms or signs of disease to be treated.

The terms “treat”, “treated”, “treating” and “treatment”, as used with respect to methods herein refer to eliminating, reducing, suppressing or ameliorating, either temporarily or permanently, either partially or completely, a clinical symptom, manifestation or progression of an event, disease or condition. Such treating need not be absolute to be useful.

### Particles

The size and charge of the particles are important for tolerance induction. While the particles will differ in size and charge based on the antigen encapsulated within them, in general, particles of the current disclosure are effective at inducing tolerance when they are between about 100 nanometers and about 1500 nanometers and have a charge of between 0 to about −100 mV. In various embodiments, the particles are 400-800 nanometers in diameter and have a charge of between about −25 mV and −70 mV. The average particle size and charge of the particles can be slightly altered in the lyophilization process, therefore, both post-synthesis averages and post-lyophilization averages are described. As used herein, the term “post-synthesis size” and “post synthesis charge” refer to the size and charge of the particle prior to lyophilization. The term “post lyophilization size” and “post lyophilization charge” refer to the size and charge of the particle after lyophilization.

In some embodiments, the particle is non-metallic. In these embodiments the particle may be formed from a polymer. In a preferred embodiment, the particle is biodegradable in an individual. In this embodiment, the particles can be provided in an individual across multiple doses without there being an accumulation of particles in the individual. Examples of suitable particles include polystyrene particles, PLGA particles, PLURIONICS stabilized polypropylene sulfide particles, and diamond particles.

Preferably the particle surface is composed of a material that minimizes non-specific or unwanted biological interactions. Interactions between the particle surface and the interstitium may be a factor that plays a role in lymphatic uptake. The particle surface may be coated with a material to prevent or decrease non-specific interactions. Steric stabilization by coating particles with hydrophilic layers such as poly(ethylene glycol) (PEG) and its copolymers such as PLURONICS® (including copolymers of poly(ethylene glycol)-bl-poly(propylene glycol)-bl-poly(ethylene glycol)) may reduce the non-specific interactions with proteins of the interstitium as demonstrated by improved lymphatic uptake following subcutaneous injections. All of these facts suggest relevance of the physical properties of the particles in terms of lymphatic uptake. Biodegradable polymers may be used to make all or some of the polymers and/or particles and/or layers. Biodegradable polymers may undergo degradation, for example, by a result of functional groups reacting with the water in the solution. The term “degradation” as used herein refers to becoming soluble, either by reduction of molecular weight or by conversion of hydrophobic groups to hydrophilic groups. Polymers with ester groups are generally subject to spontaneous hydrolysis, e.g., polylactides and polyglycolides.

Particles of the present disclosure may also contain additional components. For example, carriers may have imaging agents incorporated or conjugated to the carrier. An example of a carrier nanosphere having an imaging agent that is currently commercially available is the Kodak X-sight nanospheres. Inorganic quantum-confined luminescent nanocrystals, known as quantum dots (QDs), have emerged as ideal donors in FRET applications: their high quantum yield and tunable size-dependent Stokes Shifts permit different sizes to emit from blue to infrared when excited at a single ultraviolet wavelength. (Bruchez, et al., Science, 1998, 281, 2013; Niemeyer, C. M Angew. Chem. Int. Ed. 2003, 42, 5796; Waggoner, A. Methods Enzymol. 1995, 246, 362; Brus, L. E. J. Chem. Phys. 1993, 79, 5566). Quantum dots, such as hybrid organic/inorganic quantum dots based on a class of polymers known as dendrimers, may be used in biological labeling, imaging, and optical biosensing systems. (Lemon, et al., J. Am. Chem. Soc. 2000, 122, 12886). Unlike the traditional synthesis of inorganic quantum dots, the synthesis of these hybrid quantum dot nanoparticles does not require high temperatures or highly toxic, unstable reagents. (Etienne, et al., Appl. Phys. Lett. 87, 181913, 2005).

Particles can be formed from a wide range of materials. The particle is preferably composed of a material suitable for biological use. For example, particles may be composed of glass, silica, polyesters of hydroxy carboxylic acids, polyanhydrides of dicarboxylic acids, or copolymers of hydroxy carboxylic acids and dicarboxylic acids. More generally, the carrier particles may be composed of polyesters of straight chain or branched, substituted or unsubstituted, saturated or unsaturated, linear or cross-linked, alkanyl, haloalkyl, thioalkyl, aminoalkyl, aryl, aralkyl, alkenyl, aralkenyl, heteroaryl, or alkoxy hydroxy acids, or polyanhydrides of straight chain or branched, substituted or unsubstituted, saturated or unsaturated, linear or cross-linked, alkanyl, haloalkyl, thioalkyl, aminoalkyl, aryl, aralkyl, alkenyl, aralkenyl, heteroaryl, or alkoxy dicarboxylic acids. Additionally, carrier particles can be quantum dots, or composed of quantum dots, such as quantum dot polystyrene particles (Joumaa et al. (2006) Langmuir 22: 1810-6). Carrier particles including mixtures of ester and anhydride bonds (e.g., copolymers of glycolic and sebacic acid) may also be employed. For example, carrier particles may comprise materials including polyglycolic acid polymers (PGA), polylactic acid polymers (PLA), polysebacic acid polymers (PSA), poly(lactic-co-glycolic) acid copolymers (PLGA or PLG; the terms are interchangeable), [rho]oly(lactic-co-sebacic) acid copolymers (PLSA), poly(glycolic-co-sebacic) acid copolymers (PGSA), polypropylene sulfide polymers, poly(caprolactone), chitosan, etc. Other biocompatible, biodegradable polymers useful in the present invention include polymers or copolymers of caprolactones, carbonates, amides, amino acids, orthoesters, acetals, cyanoacrylates and degradable urethanes, as well as copolymers of these with straight chain or branched, substituted or unsubstituted, alkanyl, haloalkyl, thioalkyl, aminoalkyl, alkenyl, or aromatic hydroxy- or di-carboxylic acids. In addition, the biologically important amino acids with reactive side chain groups, such as lysine, arginine, aspartic acid, glutamic acid, serine, threonine, tyrosine and cysteine, or their enantiomers, may be included in copolymers with any of the aforementioned materials to provide reactive groups for conjugating to antigen peptides and proteins or conjugating moieties. Biodegradable materials suitable for the present invention include diamond, PLA, PGA, polypropylene sulfide, and PLGA polymers. Biocompatible but non-biodegradable materials may also be used in the carrier particles of the invention. For example, non-biodegradable polymers of acrylates, ethylene-vinyl acetates, acyl substituted cellulose acetates, non-degradable urethanes, styrenes, vinyl chlorides, vinyl fluorides, vinyl imidazoles, chlorosulphonated olefins, ethylene oxide, vinyl alcohols, TEFLON® (DuPont, Wilmington, Del.), and nylons may be employed.

In certain embodiments, the particle is a co-polymer having a molar ratio from about 80:20 to about 100:0. Suitable co-polymer ratio of present immune modified particles may be 25:75, 30:70, 35:65, 40:60, 45:55, 50:50, 55:45, 60:40, 65:35, 70:30, 75:25, 80:20, 81:19, 82:18, 83:17, 84:16, 85:15, 86:14, 87:13, 88:12, 89:11, 90:10, 91:9, 92:8, 93:7, 94:6, 95:5, 96:4, 97:3, 98:2, 99:1, or 100:0. In certain embodiments, the particle is a PLURONICS stabilized polypropylene sulfide particle, a polyglycolic acid particle (PGA), a polylactic acid particle (PLA), or a poly(lactic-co-glycolic acid) particle. In certain embodiments, the particle has a copolymer ratio of polylactic acid/polyglycolic acid 80:20: polylactic acid/polyglycolic acid 90:10, or polylactic acid: polyglycolic acid/50:50. In various embodiments, the particle is a poly(lactic-co-glycolic acid) particle and has a copolymer ratio of about 50:50 polylactic acid:polyglycolic acid.

It is contemplated that the particle may further comprise a surfactant. The surfactant can be anionic, cationic, or nonionic. Surfactants in the poloxamer and poloaxamines family are commonly used in particle synthesis. Surfactants that may be used, include, but are not limited to PEG, Tween-80, gelatin, dextran, pluronic L-63, PVA, PAA, methylcellulose, lecithin, DMAB and PEMA. Additionally, biodegradable and biocompatible surfactants including, but not limited to, vitamin E TPGS (D-a-tocopheryl polyethylene glycol 1000 succinate), poly amino acids (e.g polymers of lysine, arginine, aspartic acid, glutamic acid, serine, threonine, tyrosine and cysteine, or their enantiomers), and sulfate polymers. In certain embodiments, two surfactants are used. For example, if the particle is produced by a double emulsion method, the two surfactants can include a hydrophobic surfactant for the first emulsion, and a hydrophobic surfactant for the second emulsion. In certain embodiments, the polypeptide antigens are encapsulated in the particles by a single-emulsion process. In a further embodiment, the polypeptide antigens are more hydrophobic. Sometimes, the double emulsion process leads to the formation of large particles which may result in the leakage of the hydrophilic active component and low entrapment efficiencies. The coalescence and Ostwald ripening are two mechanisms that may destabilize the double-emulsion droplet, and the diffusion through the organic phase of the hydrophilic active component is the main mechanism responsible of low levels of entrapped active component. In some embodiments, it may be beneficial to reduce the nanoparticle size. One strategy to accomplish this is to apply a second strong shear rate. The leakage effect can be reduced by using a high polymer concentration and a high polymer molecular mass, accompanied by an increase in the viscosity of the inner water phase and in increase in the surfactant molecular mass. In certain embodiments, the particles encapsulating antigens are manufactured by nanoprecipitation, co-precipitation, inert gas condensation, sputtering, microemulsion, sol-gel method, layer-by-layer technique or ionic gelation method. Several methods for manufacturing nanoparticles have been described in the literature and are incorporated herein by reference (Sánchez, Mejía, and Orozco 2020; Zielińska et al. 2020).

### Antigens

An antigen refers to a discreet portion of a molecule, such as a polypeptide or peptide sequence, a 3-D structural formation of a polypeptide or peptide, a polysaccharide or polynucleotide that can be recognized by a host immune cells. Antigen-specific refers to the ability of a subject's host cells to recognize and generate an immune response against an antigen alone, or to molecules that closely resemble the antigen, as with an epitope or mimotope.

“Anergy,” “tolerance,” or “antigen-specific tolerance” refers to insensitivity of T cells to T cell receptor-mediated stimulation. Such insensitivity is generally antigen-specific and persists after exposure to the antigenic peptide has ceased. For example, anergy in T cells is characterized by lack of cytokine production, e.g., IL-2. T-cell anergy occurs when T cells are exposed to antigen and receive a first signal (a T cell receptor or CD-3 mediated signal) in the absence of a second signal (a costimulatory signal). Under these conditions, re-exposure of the cells to the same antigen (even if re-exposure occurs in the presence of a costimulatory molecule) results in failure to produce cytokines and subsequently failure to proliferate. Thus, a failure to produce cytokines prevents proliferation. Anergic T cells can, however, proliferate if cultured with cytokines (e.g., IL-2).

It is contemplated that the tolerizing therapy described herein is antigen-specific. For example, TIMPs administered in tolerizing therapy encapsulate one or more antigens associated with said tolerizing therapy and associated disease or condition being treated. In various embodiments, the antigen is an autoimmune antigen, a transplant antigen, an allergen, an enzyme replacement therapy, a protein therapeutic, and/or a gene therapy vector or viral vector.

Exemplary antigens include myelin basic protein, acetylcholine receptor, endogenous antigen, myelin oligodendrocyte glycoprotein (MOG), myelin basic protein (MBP), proteolipid protein (PLP), myelin associated glycoprotein (MAG), cyclic nucleotide phosphohydrolase, pancreatic beta-cell antigen, insulin, proinsulin, islet-specific glucose-6-phophatase catalytic subunit-related protein (IGRP), glutamic acid decarboxylase (GAD), collagen type 11, human cartilage gp39, fp130-RAPS, fibrillarin, small nucleolar protein, thyroid stimulating factor receptor, histones, glycoprotein gp70, pyruvate dehydrogenase dehydrolipoamide acetyltransferase (PCD-E2), hair follicle antigen, aqua porin 4, Desmoglein 1, Desmoglein 3, nicotinic acetylcholine receptor, gliadin, ADAMTS13, GPIlb/GPIlla, CYP2D6, BP180, NC16, BP230, Ro60, MPO, thyroid stimulating hormone receptor, and human tropomyosin isoform 5, Bahia grass pollen (BaGP), peach allergen, milk allergens, celery allergens, nut allergens, tree-nut allergen, bovine serum albumin, Hazelnut allergens, ovalbumin, egg allergen, peanut allergens, fish allergens, shellfish allergens, dust mite, cat allergen, dog allergen, pollen allergen, bee venom, Japanese cedar pollen, an enzyme replacement therapy, a therapeutic protein, and a viral vector.

Over 15 peanut allergens are officially recognized by the WHO/IUIS Allergen Nomenclature Sub-Committee (www.allergen.org), Ara h1 to Ara h18, including Ara h1, Ara h2, Ara h3, Ara h5, Ara h6, Ara h7, Ara h8, Ara h9, Ara h10, Ara h11, Ara h12, Ara h13, Ara h14, Ara h15, Ara h16, Ara h17 and Ara h18. Peanut allergens can be classified into different groups based on their architecture (e.g., trimer, monomer, cupin, albumin, prolamin, profilin, oleosins, defensins, vincillin, Nonspecific lipid transfer proteins (nsLTPs)) based on Ara h1, h2, h3, h5, h6 and h8, and each of these groups possesses a different degree of allergenic potency (Ozias-Akins et al., Allergy 74:888-898, 2019). Known peanut allergens include those derived from Arachis hypogaea Ara h1, Ara h2, Ara h3, Ara h5, Ara h6, Ara h7, and Ara h8 and Ara h18. See e.g., UNIPROT Database No. E5G076 showing the Ara h1 polypeptide sequence (SEQ ID NO: 1), UNIPROT Database No. A0A445BY15 for Ara h2 polypeptide (SEQ ID NO: 2), UNIPROT Database No. E5G077 for Ara h3 polypeptide (SEQ ID NO: 3) (see also UNIPROT Database No. O82580 (SEQ ID NO: 4) and Q9SQH7 (SEQ ID NO: 5) for Ara h3 isoallergens 1 and 2 (formerly Ara h4), respectively), UNIPROT Database No. L7QH52 for Ara h5 polypeptide (SEQ ID NO: 6), UNIPROT Database No. A5Z1R0 for Ara h6 polypeptide (SEQ ID NO: 7), UNIPROT Database No. B4XID4 for Ara h7 polypeptide (SEQ ID NO: 8), UNIPROT Database No. Q6VT83 for Ara h8 polypeptide sequence (SEQ ID NO: 9), Ara h9, isoallergen1 and 2, UNIPROT Database No. B6CEX8 and B6CG41, (SEQ ID NO: 10 and 11) respectively; Ara h10, isoallergen 1 and 2, UNIPROT Database No. Q647G5 and Q647G4, (SEQ ID NO: 12 and 13) respectively; Ara h11, isoallergen 1 and 2, UNIPROT Database No. Q45W87 and Q45W86, (SEQ ID NO: 14 and 15) respectively; Ara h12 UNIPROT Database No. B3EWP3 (SEQ ID NO: 16); Ara h13, isoallergen 1 and 2, UNIPROT Database No. B3EWP4 and COHJZ1, (SEQ ID NO: 17 and 18) respectively; Ara h14, isoallergen 1, 2, and 3, UNIPROT Database No. Q9AXI1, Q9AXI0 and Q6J1J8, (SEQ ID NO: 19-21) respectively; Ara h15, UNIPROT Database No. Q647G3 (SEQ ID NO: 22); Ara h16, UNIPROT Database No. A0A509ZX51 (SEQ ID NO: 23); Ara h17, UNIPROT A Database No. 0A510A9S3 (SEQ ID NO: 24); and Ara h18, UNIPROT Database No. A0A444XS96 (SEQ ID NO: 25).

In certain embodiments, one, two, three, or a higher number of antigens or antigenic peptides are used in the TIMPs. In certain embodiments, the one or more antigens are encapsulated in the TIMP by covalent linkage to the interior surface of the particle (See e.g., US Patent Publication US20190282707, herein incorporated by reference). In certain embodiments, it is contemplated that sequences of two or more antigens are linked in a fusion protein and encapsulated within a TIMP described herein. Methods for making TIMP with linked epitopes are described in US Patent Publication US20190365656, herein incorporated by reference.

Enzyme replacement therapy (ERT) is often used to treat genetic diseases, e.g., lysosomal storage disorders or hemophilia, in which a protein or enzyme is dysfunctional in a subject and administration of exogenous protein can reduce symptoms of a disorder being treated. However, in certain situations, ERT can induce antibodies or other immune reactions against the protein being administered. As such, a method to reduce these possible immune effects include administration of TIMPs containing proteins used in ERT. Exemplary enzyme replacement therapy proteins to be encapsulated in a TIMP include Agalsidase beta, Agalsidase alfa, Imiglucirase, Taliglucirase alfa, Velaglucerase alfa, Alglucerase, Sebelipase alpha, Laronidase, Idursulfase, Elosulfase alpha, Galsulfase, Alglucosidase alpha, Factor VII, Factor VIII, Factor IX, Acetylgalactosamine 4-sulfate, Iduronidase, Alglucerase, or Glucocerebrosidase.

Exemplary protein therapeutics include a recombinant protein selected from the group consisting of erythropoietin, insulin, human growth hormone, follicle-stimulating hormone, granulocyte colony-stimulating factor, tissue plasminogen activator, insulin-like growth factor, uricase, kynurinase, L-arginine deaminase, arginase, methionine-γ-lyase, asparaginase, an amino acid degrading enzyme, a gluten degrading enzyme, a nucleotide degrading enzyme, IFN-γ, IL-2, IL-12, or IL-15.

In certain embodiments, the protein therapeutic is an antibody, e.g., a monoclonal or a polyclonal antibody. It is also contemplated that the antibody is mono-specific, bi-specific, tri-specific, or bi-specific T-cell engager. In various embodiments the antibody targets receptor tyrosine kinase (RTK), EGFR, VEGF, VEGFR, PDGF, PDGFR, HER2/Neu, ER, PR, TGF-ß1, TGF-β2, TGF-β3, SIRP-α, PD-1, PD-L1, CTLA-4, CD3, CD25, CD19, CD20, CD39, CD47, CD73, FAP, IL-1B, IL-12, IL-2R, IL-15, IL-15R, IL-23, IL-33, IL-2R, IL-4Rα, T-cells, B-cells, NK cells, macrophages, monocytes, and/or neutrophils. In various embodiments, the antibody is selected from the group consisting of abciximab, adalimumab, alemtuzumab, avelumab, azetolizumab, basiliximab, bevacizumab, bezlotoxumab, blinatumomab, canakinumab, certolizumab, cetuximab, daclizumab, denosumab, durvalumab, efalizumab, emicizumab, etokimab, golimumab, ipilimumab, ixekizumab, infliximab, natalizumab, nivolumab, olaratumab, omalizumab, ofatimumab, palivizumab, panitumumab, pembrolizumab, ramucirumab, rituximab, tocilizumab, trastuzumab, tremelimumab, secukinumab, ustekinumab, and vedolizumab.

Some therapeutics, such as gene therapy or cancer vaccines, are delivered using viral vectors. However, many individuals naturally carry antibodies against certain viral vectors, and anti-virus antibodies and other immune activity against the viral vectors can arise as a result of treatment. As such, tolerance to the viral vectors can improve therapies that employ viral vectors. In certain embodiments, the TIMP comprises all or part of a viral vector. Exemplary viruses useful as viral vectors include, but not limited to, adeno-associated virus (AAV), herpes simplex virus, lentivirus, retrovirus, alphavirus, flavivirus, rhabdovirus, measles virus, Newcastle disease virus, poxvirus, vaccinia virus, modified Ankara virus, vesicular stomatitis virus, picornavirus, tobacco mosaic virus, potato virus x, comovirus or cucumber mosaic virus. In various embodiments, the virus is an oncolytic virus. In various embodiments the virus is a chimeric virus, a synthetic virus, a mosaic virus or a pseudotyped virus. In various embodiments, the AAV vector is AAV1, AAV2, AAV3, AAV4, AAV5, AAV6, AAV7, AAV8, AAV9, AAV12, Anc80 or combinations thereof.

### Methods of Use

The present methods are useful to monitor and track the induction of, and more importantly, the maintenance of tolerance in a subject undergoing tolerizing therapy. The present methods are useful to determine an immune signature in an individual undergoing treatment and establishing an immune signature indicates if the subject has lasting immune tolerance, or if antigen specific tolerance is waning and additional treatment with the tolerizing therapy is needed. The methods are also useful in informing dosing of individuals receiving therapy, such that the immune signature may indicate that a higher or lower dose of tolerizing therapeutic is warranted in in the subject.

In various embodiments, the subject is undergoing treatment with an immune tolerizing therapy. In various embodiments, the subject is undergoing treatment with a desensitization therapy. Treatment includes oral immunotherapy (OIT), subcutaneous immunotherapy (SCIT), sublingual immunotherapy (SLIT), and immune tolerizing nanomedicine. In various embodiments, the treatment is an immune tolerizing nanomedicine. In various embodiments, the immune tolerizing nanomedicine is tolerizing immune modulating particles (TIMPs).

In various embodiments, TIMPs are administered alone or in combination with one or more additional therapeutics. In various embodiments, the additional therapeutic is an inhibitor of IgE, an inhibitor of basophil activation, an inhibitor of mast cell activation, an antihistamine, or a small molecule or biological therapeutic. In various embodiments, the additional therapeutic inhibits IgE. In various embodiments, the additional therapeutic inhibits basophil activation. In various embodiments, the additional therapeutic inhibits mast cell activation. In various embodiments, the additional therapeutic is a biologic or a small molecule. In various embodiments, the additional therapeutic is an anti-IgE antibody, an anti-IL-4Rα antibody, an anti-IL13 antibody, an anti-IL-33 antibody, an antihistamine, a steroid, a corticosteroid, a leukotriene modifier, or an nonsteroid anti-inflammatory drug (NSAID).

In various embodiments, the additional therapeutic is an antihistamine. In various embodiments, the antihistamine is a first generation antihistamine. In various embodiments, the antihistamine is a second generation antihistamine. In various embodiments, the antihistamines are selected from the group consisting of brompheniramine, carbinoxamine maleate, chlorpheniramine, clemastine, diphenhydramine, hydroxyzine, triprolidine, azelastine, cetirizine, desloratadine, fexofenadine, levocetrizine, doxylamine, ebastine, embramine, epinephrine, fexofenadine, loratadine, and olopatadine.

In various embodiments, the additional therapeutic is a steroid. In various embodiments, the steroid is selected from the group consisting of beclomethasone, ciclesonide, fluticasone furoatr, mometasone, budenoside, fluticasone, triamcinolone, and loteprednol.

In various embodiments, the additional therapeutic is a corticosteroid. In various embodiments, the corticosteroid is selected from the group consisting of cortisone, prednisone, prednisolone, methylprednisolone, dexamethasone, betamethasone, and hydrocortisone.

In various embodiments, the additional therapeutic is a nonsteroid anti-inflammatory drug (NSAID). In various embodiments the NSAID is a non-selective NSAID. In various embodiments the NSAID is a COX-2 selective NSAID. In various embodiments the NSAID is a COX-1 selective NSAID. In various embodiments the NSAID is a prostaglandin synthase inhibitor. In various embodiments, the NSAID is selected from the group consisting of diclofenac, diclofenac potassium, diclofenac sodium, diflunisal, etodolac, flurbiprofen, fenoprofen, fenoprofen calcium, ketorolac, ketorolac tromethamine, ketoprofen, tolmetin, tolmetin sodium, aspirin, ibuprofen, naproxen, indomethacin, indomethacin sodium, sulindac, felbinac, piroxicam, mefenamic acid, meclofenamate sodium, meloxicam, nabumetone, oxaprozin, piroxicam, celecoxib, etodolac, etoricoxib, lumiracoxib, rofecoxib, and valdecoxib.

In various embodiments, the additional therapeutic is a leukotriene modifier. In various embodiments the leukotriene modifier is an antileukotriene. In various embodiments the leukotriene modifier is a leukotriene receptor antagonist. In various embodiments the leukotriene modifier is a leukotriene synthesis inhibitor. In various embodiments the leukotriene modifier is selected from the group consisting of montelukast, zileuton, and zafirlukast.

To assess tolerance, biological samples are obtained from the subject before and during therapy and assayed for the various parameters of tolerance described herein. Biological samples include whole-blood, peripheral blood, peripheral blood mononuclear cells (PBMCs), serum, plasma, urine, cerebrospinal fluid (CSF), stool, a tissue biopsy, and/or a bone-marrow biopsy. In various embodiments, the assay of the biological sample(s) includes analyzing levels of, and or presence or absence of, cell-surface proteins, extracellular proteins, intracellular proteins, nucleic acids, metabolites, and/or combinations thereof.

Cells assayed from the biological sample include immune cells, non-immune cells, and/or combinations thereof. Immune cells include innate immune cells, adaptive immune cells, and/or combinations thereof. Innate immune cells assayed from the biological sample(s) are antigen-presenting cells (APCs). Exemplary innate immune cells assayed from the biological sample include monocytes, macrophages, neutrophils, granulocytes, dendritic cells, mast cells, eosinophils, basophils, and/or combinations thereof. Adaptive immune cells assayed from the biological sample(s) include effector immune cells, such as T-cells, B-cells, NK cells, NK-T cells, and/or combinations thereof. In various embodiments, the T cells are TH1 cells, TH2a cells, Treg cells, and Tr1 cells.

In certain embodiments, the cells assayed from the biological sample(s) are epithelial cells, stromal cells, endothelial cells, fibroblasts, pericytes, adipocytes, mesenchymal stem cells, hematopoietic stem cells, hematopoietic progenitor cells, liver sinusoidal endothelial cells (LSECs), and/or Kupffer cells.

The immune tolerance signature of a subject is generated using one or more of the following parameters assayed from one or more biological samples obtained from the subject and stimulated in vivo and/or ex vivo:
a. proportion of effector T cells in the total T cell population,
b. proportion of Treg cells in the total T cell population,
c. proportion of effector B cells in the total B cell population,
d. levels and/or ratios of specific IgG, IgA, IgM, and/or IgE,
e. levels of inflammatory cytokines and chemokines,
f. levels of anti-inflammatory cytokines and chemokines,
g. levels of inflammatory metabolites, and
h. levels of anti-inflammatory metabolites.

The immune tolerance signature is indicative of maintenance of immune tolerance if 1, 2, 3, 4, 5, 6, 7, or 8 parameters listed in (a)-(h) above indicate maintenance of immune tolerance. In various embodiments, the immune tolerance signature is indicative of maintenance of immune tolerance if at least 2/8 parameters listed in (a)-(h) indicate maintenance of immune tolerance. In various embodiments, the subject is determined to not require treatment with TIMPs if 1, 2, 3, 4, 5, 6, 7, or 8 parameters listed in (a)-(h) above indicate maintenance of immune tolerance. In various embodiments, the subject is determined to not require treatment with TIMPs if at least 3/8 parameters listed in (a)-(h) above indicate maintenance of immune tolerance.

The immune tolerance signature of a subject generated using one or more parameters described herein indicates weakening and/or absence of immune tolerance prior to or after treatment with tolerizing therapy, e.g., TIMPs, if:
a. the proportion of effector T cells in the total T cell population is between 5%-100% (e.g., about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, inclusive of all values and ranges between these values), and/or
b. the proportion of Treg cells in the total T cell population is between 1-3%, and/or
c. the proportion of effector B cells in the total B cell population is between 5%-100% (e.g. about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, inclusive of all values and ranges between these values), and/or
d. the levels and/or ratios of IgG, IgA, IgM, and/or IgE are increased by about 5%-100% (e.g. about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, inclusive of all values and ranges between these values), 10-95%, 15-90%, 20-85%, 25-75%, 30-70%, 35-65%, 40-60%, 45-55%, or 50% or by about 2-100-fold (e.g., about 2, 5, 10, 15, 20, 25, 30, 35, 40, 45, 50, 55, 60, 65, 70, 75, 80, 85, 90, 95, or 100-fold inclusive of all values and ranges between these values) relative to a healthy subject and/or one or more baseline measurements taken from the subject during treatment, and/or
e. levels of inflammatory cytokines/chemokines are increased by about 5%-100% (e.g. about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, inclusive of all values and ranges between these values), 10-95%, 15-90%, 20-85%, 25-75%, 30-70%, 35-65%, 40-60%, 45-55%, or 50% or by about 2-100-fold (e.g., about 2, 5, 10, 15, 20, 25, 30, 35, 40, 45, 50, 55, 60, 65, 70, 75, 80, 85, 90, 95, or 100-fold inclusive of all values and ranges between these values) relative to a healthy subject and/or one or more baseline measurements taken from the subject during treatment, and/or
f. levels of anti-inflammatory cytokines and chemokines are decreased by about 5%-100% (e.g. about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, inclusive of all values and ranges between these values), 10-95%, 15-90%, 20-85%, 25-75%, 30-70%, 35-65%, 40-60%, 45-55%, or 50% or by about 2-100-fold (e.g., about 2, 5, 10, 15, 20, 25, 30, 35, 40, 45, 50, 55, 60, 65, 70, 75, 80, 85, 90, 95, or 100-fold inclusive of all values and ranges between these values) relative to a healthy subject and/or one or more baseline measurements taken from the subject during treatment, and/or
g. levels of inflammatory metabolites are increased by about 5%-100% (e.g. about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, inclusive of all values and ranges between these values), 10-95%, 15-90%, 20-85%, 25-75%, 30-70%, 35-65%, 40-60%, 45-55%, or 50% or by about 2-100-fold (e.g., about 2, 5, 10, 15, 20, 25, 30, 35, 40, 45, 50, 55, 60, 65, 70, 75, 80, 85, 90, 95, or 100-fold inclusive of all values and ranges between these values) relative to a healthy subject and/or one or more baseline measurements taken from the subject during treatment, and/or
h. levels of anti-inflammatory metabolites are decreased by about 5%-100% (e.g. about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, inclusive of all values and ranges between these values), 10-95%, 15-90%, 20-85%, 25-75%, 30-70%, 35-65%, 40-60%, 45-55%, or 50% or by about 2-100-fold (e.g., about 2, 5, 10, 15, 20, 25, 30, 35, 40, 45, 50, 55, 60, 65, 70, 75, 80, 85, 90, 95, or 100-fold inclusive of all values and ranges between these values) relative to a healthy subject and/or one or more baseline measurements taken from the subject during treatment.

It is contemplated that the one or more biological samples used in the methods are collected from the subject undergoing tolerizing therapy 1-7 days, 1-4 weeks, and/or 1-12 months prior to administration of immune tolerizing therapy. In various embodiments, the one or more biological samples are collected 1-7 days, 1-4 weeks, and/or 1-12 months after administration of the immune tolerizing therapy. In various embodiments, the one or more biological samples are collected every 1-7 days, every 1-4 weeks, and/or every 1-12 months after administration of the immune tolerizing therapy. In various embodiments, the one or more biological samples are collected at intervals of 1-7 days, every 1-4 weeks, and/or every 1-12 months after administration of the immune tolerizing therapy. In various embodiments, the samples are collected every week, 2 weeks, 4 weeks, 1 month, 2 months, 3 months, 4 months, 5 months, 6 months, 9 months or 12 months.

### Screening Methods

Methods of screening for cell types, cytokines, nucleic acids, or other measures of tolerance from a subject undergoing tolerizing therapy as described herein are known in the art. Methods of assessing tolerance are done using such techniques as flow cytometry, Mass Cytometry (CyTOF), ELISA, ELISPOT, in vitro/ex vivo cell stimulation assays (including, but not limited to, cell proliferation assays, basophil activation test (BAT), macrophage stimulation assays), measuring autoantibodies or measuring Ig serotype, e.g., by ImmunoCap assay.

One aspect of a subject's immune tolerance status, and immune signature, is determined by analyzing one or more cell-surface proteins from a biological sample(s). In various embodiments, the cell-surface proteins include CD1c, CD2, CD3, CD4, CD5, CD8, CD9, CD10, CD11b, CD11c, CD14, CD15, CD16, CD18, CD19, CD20, CD21, CD22, CD23, CD24, TACI, CD25, CD27, CD28, CD30, CD30L, CD31, CD32, CD32b, CD34, CD33, CD38, CD39, CD40, CD40-L, CD41b, CD42a, CD42b, CD43, CD44, CD45, CD45RA, CD47, CD45RA, CD45RO, CD48, CD52, CD55, CD56, CD58, CD61, CD66b, CD69, CD70, CD72, CD79, CD68, CD84, CD86, CD93, CD94, CD95, CRACC, BLAME, BCMA, CD103, CD107, CD112, CD120a, CD120b, CD123, CD125, CD127, CD134, CD135, CD140a, CD141, CD154, CD155, CD160, CD161, CD163, CD172a, XCR1, CD203c, CD204, CD206, CD207 CD226, CD244, CD267, CD268, CD269, CD355, CD358, CRTH2, NKG2A, NKG2B, NKG2C, NKG2D, NKG2E, NKG2F, NKG2H, KIR2DL1, KIR2DL2, KIR2DL3, KIR2DL5A, KIR2DL5B, KIR3DL1, KIR3DL2, KIR3DL3, KIR3DL4, KIR2DS1, KIR2DS2, KIR2DS3, KIR2DS4, KIR2DS5, DAP12, KIR3DS, NKp44, NKp46, TCR, BCR, Integrins, FcBERI, MHC-I, MHC-II, IL-1R, IL-2Rα, IL-2Rβ, IL-2Rγ, IL-3Rα, CSF2RB, IL-4R, IL-5Rα, CSF2Rβ, IL-6Rα, gp130, IL-7Rα, IL-9R, IL-10R, IL-12Rβ1, IL-12Rβ2, IL-13Rα1, IL-13Rα2, IL-15Rα, IL-21R, IL-23R, IL-27Rα, IL-31Rα, OSMR, CSF-1R, cell-surface IL-15, IL-10Rα, IL-10Rβ, IL-20Rα, IL-20Rβ, IL-22Rα1, IL-22Rα2, IL-22Rβ, IL-28RA, PD-1, PD-1H, BTLA, CTLA-4, PD-L1, PD-L2, 2B4, B7-1, B7-2, B7-H1, B7-H4, B7-DC, DR3, LIGHT, LAIR, LTα1β2, LTβR, TIM-1, TIM-3, TIM-4, TIGIT, LAG-3, ICOS, ICOS-L, SLAM, SLAMF2, OX-40, OX-40L, GITR, GITRL, TL1A, HVEM, 41-BB, 41BB-L, TL-1A, TRAF1, TRAF2, TRAF3, TRAF5, BAFF, BAFF-R, APRIL, TRAIL, RANK, AITR, TRAMP, CCR1, CCR2, CCR3, CCR4, CCR5, CCR6, CCR7, CCR8, CCR9, CCR10, CCR11, CXCR1, CXCR2, CXCR3, CXCR4, CXCR5, CXCR6, CXCR7, CLECL9a, DC-SIGN, IGSF4A, SIGLEC, EGFR, PDGFR, VEGFR, FAP, α-SMA, FAS, FAS-L, FC, ICAM-1, ICAM-2, ICAM-3, ICAM-4, ICAM-5, PECAM-1, MICA, MICB, UL16, ULBP1, ULBP2, ILBP3, ULBP4, ULBP5, ULBP6, MULT1, RAE1 α,β,γ,δ, and ε, H60a, H60b, H60c, GPR15, ST2, and/or combinations thereof. Integrins include α1, α2, αIIb, α3, α4, α5, α6, α7, α8, α9, α10, α11, αD, αE, αL, αM, αV, αX, β1, β2, β3, β4, β5, β6, β7, β8 and/or combinations thereof. TCR include α, β, γ, δ, ε, ζ chains and/or combinations thereof. Several methods have been described in the literature for assaying of cell-surface protein expression, including Flow Cytometry and Mass Cytometry (CyTOF).

In certain embodiments, the subject's tolerance status is determined by analyzing nucleic acids from the biological sample(s). In various embodiments, the nucleic acids are DNA and/or RNA, including, but not limited to, single stranded DNA, double stranded DNA, mRNA, rRNA, tRNA, siRNA, miRNA, long non-coding RNAs (long ncRNAs, lncRNA), and non-coding RNA (ncRNA), and mitochondrial RNA. In various embodiments, the subject's immune tolerance status is determined by assaying gene expression from the biological sample(s). In various embodiments, the immune tolerance status is determined by assaying gene expression associated with immune function, an antibody, foreign body response, metabolism, apoptosis, cell death, necrosis, ferroptosis, autophagy, cell migration, endocytosis, phagocytosis, pinocytosis, tight-junction regulation, cell adhesion, differentiation, and/or combinations thereof. In various embodiments, the immune tolerance status is determined by assaying gene expression associated with immune suppression. In various embodiments, the immune tolerance status is determined by assaying gene expression associated with immune activation. In various embodiments, the immune tolerance status is determined by assaying gene expression associated with immune regulatory functions. In various embodiments, nucleic acid analysis is used to generate an immune tolerance signature. Several methodologies have been described in the literature for high-throughput gene expression analysis including RNA sequencing (RNA-seq), single-cell RNA sequencing (scRNA-seq), exome sequencing, and microarray-based analyses.

The biological sample is optionally assayed after in vivo and/or ex vivo stimulation with one or more stimuli such as an antigen, an allergen, and one or more activating agents. It is contemplated that the T cells, B cells, and immunoglobulins used in the assay are antigen specific. Exemplary T cells include effector memory T cells, antigen specific T cells, activated antigen specific T cells, Th1 cells, pathogenic Th2a+ cells, Th17 cells, T follicular helper (TFH) cells, Th0 cells, or other antigen specific T cells. B cells include effector B cells, memory B cells, plasma B cells, and Breg cells. In certain embodiments, T cells are identified based on the expression of proteins described in Table A.

TABLE A

| Cell Type | Expression Marker |
|---|---|
| Effector memory (EM) | CD45RA^{LO} CD4⁺ |
| T cell | |
| Disease specific T cell | CD45RA^{LO} CD4⁺ CD154⁺ CD137⁺ |
| Activated disease | CD38⁺ |
| specific T cell | |
| Pathogenic T_{H}2a+ cell | CD45RA^{LO} CD4⁺ CD154⁺ CD137⁺ CXCR5⁻ |
| | CRTH2^{+/-} CD27⁻ CD200R⁺ CD161⁺ |
| T_{H}1 cell | CXCR5⁻ CRTH2⁻ CXCR3⁺ CCR6⁻ |
| T_{H}17 cell | CXCR5⁻ CRTH2⁻ CXCR3⁻ CCR6⁺ |
| TFH cell | CD45RA^{LO} CD4⁺ CXCR5⁺ |
| T_{reg} | CD45RA^{LO} CD4⁺ CD154⁻ CD127⁻ CD25⁺ |
| | TIGIT⁺ CD137⁺ GARP^{+/-} OX40^{+/-} |
| T_{H}0 cell | CXCR5⁻ CRTH2⁻ CXCR3⁻ CCR6⁻ CCR4⁻ |
| Activated antigen | CD8⁺ CD137⁺ CD69⁺ CD355⁺ |
| specific CD8 T cell | |
| Gut homing T cell | CD4⁺ α4β7⁺ and CD8⁺ αEβ7⁺ |
| Brain homing T cells | CD45RO⁺ CCR7⁺ CD27⁺ and/or CD45RO⁺ |
| | CCR7⁺ CCR5⁺ CXCR3⁺ CD27⁺ |
| Exhausted T cells | CD4⁺ TIGIT⁺ KLRG1⁺ PD-1⁺ |

Measurement of cell types and cytokines in a sample can be done by flow cytometry. For example, PBMCs are activated ex vivo for 24-48 hours with specific antigens, stained, and analyzed by flow cytometry to determine the different cell types, e.g., Teff, Treg, Th1, B cells, etc. and cytokines IL5, IFNγ,

BAT assay is performed from fresh blood following ex vivo activation by antigen at multiple concentrations. Analysis is performed to provide the effective concentration at 50% of maximal basophil activation (EC50) (CD203c+/CD63+/−basophil activation).

Measurement of immunoglobulin isotype can be carried out by ImmunoCap assay.

### Pharmaceutical Formulations

Pharmaceutical compositions of the present disclosure containing the TIMP described herein and n antigen may contain pharmaceutically acceptable carriers or additives depending on the route of administration. Examples of such carriers or additives include water, a pharmaceutical acceptable organic solvent, collagen, polyvinyl alcohol, polyvinylpyrrolidone, a carbox-yvinyl polymer, carboxymethylcellulose sodium, polyacrylic sodium, sodium alginate, water-soluble dextran, carboxymethyl starch sodium, pectin, methyl cellulose, ethyl cellulose, xanthan gum, gum Arabic, casein, gelatin, agar, diglycerin, glycerin, propylene glycol, polyethylene gly-col, Vaseline, paraffin, stearyl alcohol, stearic acid, human serum albumin (HSA), mannitol, sor-bitol, lactose, a pharmaceutically acceptable surfactant and the like. Additives used are chosen from, but not limited to, the above or combinations thereof, as appropriate, depending on the dosage form of the present disclosure.

Formulation of the pharmaceutical composition will vary according to the route of administration selected (e.g., solution, emulsion). An appropriate composition comprising the therapeutic to be administered can be prepared in a physiologically acceptable vehicle or carrier. For solutions or emulsions, suitable carriers include, for example, aqueous or alcoholic/aqueous solutions, emulsions or suspensions, including saline and buffered media. Parenteral vehicles can include sodium chloride solution, Ringer's dextrose, dextrose and sodium chloride, lactated Ringer's or fixed oils. Intravenous vehicles can include various additives, preservatives, or fluid, nutrient or electrolyte replenishers.

A variety of aqueous carriers, e.g., sterile phosphate buffered saline solutions, bacteriostatic water, water, buffered water, 0.4% saline, 0.3% glycine, and the like, and may include other proteins for enhanced stability, such as albumin, lipoprotein, globulin, etc., subjected to mild chemical modifications or the like.

Therapeutic formulations of the inhibitors are prepared for storage by mixing the inhibitor having the desired degree of purity with optional physiologically acceptable carriers, excipients or stabilizers (Remington's Pharmaceutical Sciences 16th edition, Osol, A. Ed. (1980)), in the form of lyophilized formulations or aqueous solutions. Acceptable carriers, excipients, or stabilizers are nontoxic to recipients at the dosages and concentrations employed, and include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl para-bens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as TWEEN™, PLURONICS™ or polyethylene glycol (PEG).

The formulations to be used for in vivo administration must be sterile. This is readily accomplished by filtration through sterile filtration membranes.

Aqueous suspensions may contain the active compound in admixture with excipients suitable for the manufacture of aqueous suspensions. Such excipients are suspending agents, for example sodium carboxymethylcellulose, methylcellulose, hydroxypropylmethylcellulose, sodium alginate, polyvinylpyrrolidone, gum tragacanth and gum acacia; dispersing or wetting agents may be a naturally-occurring phosphatide, for example lecithin, or condensation products of an alkylene oxide with fatty acids, for example polyoxyethylene stearate, or condensation products of ethylene oxide with long chain aliphatic alcohols, for example heptadecaethyl-eneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more preservatives, for example ethyl, or n-propyl, p-hydroxybenzoate.

The TIMP comprising antigen as described herein can be lyophilized for storage and reconstituted in a suitable carrier prior to use.

Solid dosage forms for oral administration include capsules, tablets, pills, powders, and granules. In such solid dosage forms, the modified particles are mixed with at least one inert, pharmaceutically acceptable excipient or carrier such as sodium citrate or dicalcium phosphate and/or a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form may also comprise buffering agents.

### Kits

As an additional aspect, the disclosure includes kits which comprise one or more compounds or compositions packaged in a manner which facilitates their use to practice methods of the disclosure. In one embodiment, such a kit includes a compound or composition described herein (e.g., a composition comprising a TIMP alone or in combination with another antibody or a third agent), packaged in a container such as a sealed bottle or vessel, with a label affixed to the container or included in the package that describes use of the compound or composition in practicing the method. Preferably, the compound or composition is packaged in a unit dosage form. The kit may further include a device suitable for administering the composition according to a specific route of administration or for practicing a screening assay. Preferably, the kit contains a label that describes use of the inhibitor compositions.

Additional aspects and details of the disclosure will be apparent from the following examples, which are intended to be illustrative rather than limiting.

### EXAMPLES

### Example 1

It is contemplated that maintenance of immunological tolerance is monitored in a subject suffering from peanut allergy who is treated or about to undergo treatment with antigen-specific tolerizing therapy consisting of TIMPs encapsulating peanut allergens (TIMP-PPE). Subjects are expected to receive two doses of TIMP-PPE one-week apart on Days 1 and 8.

Briefly, the immune tolerance status of the subject is determined by obtaining one or more whole blood samples from the subject pre-dose on the day of the first TIMP-PPE administration (Day 1), 14 days after administration of the second dose, and then at every 90 days post-second dose (e.g., Days 90, 180, 270, and 360 post-second dose). Whole blood is processed to isolate PBMCs, basophils, neutrophils, plasma, and serum for downstream analyses.

The following indicators of immune tolerance status can be examined from assay of PBMCs isolated from one or more blood samples collected from the subject and stimulated ex vivo with purified antigenic peanut proteins, and can be measured for example, by flow cytometry:
a. Proportion of Th2a+ T cells (Th2a+ cells/total peanut reactive T cells) determined by flow cytometry. Th2a+ cells are defined as CRTH2+/CD161+/CD154+/CD27−. Total peanut reactive cells defined as CRTH2−/CD161+/CD154+/CD27−.
b. Proportion of activated peanut-specific T cells (activated peanut-specific T cells/un activated peanut reactive T cells) determined by flow cytometry. Activated peanut reactive T cells are defined as CD154+/CD38+. Un-activated peanut-specific T cells defined as CD154+.
c. Frequency of T regulatory cell population (CD4+/CD25+/FoxP3+/Helios+/IL-10+) determined by flow cytometry. Multicolor flow analysis is performed to provide the proportion of peanut specific T regulatory cells (peanut specific T regulatory cells/peanut specific CD4+ effector memory cells).
d. Ratio of IL-5 to IFN-γ in the PBMC culture supernatant following ex vivo stimulation with peanut antigen proteins, e.g., as detected by Luminex 200.

The following indicators of immune tolerance status can be examined from the assay of basophils isolated from one or more blood samples collected from the subject and stimulated ex vivo with purified antigenic peanut proteins: Proportion of activated CD203+/CD63+ basophils after of ex vivo stimulation with purified antigenic peanut proteins using a basophil activation test (BAT) (Santos and Lack 2016) and Effective concentration at 50% of maximal basophil activation (EC50) after ex vivo stimulation with purified antigenic peanut protein measured using a basophil activation test where activated basophils are CD203+/CD63+/−. Analysis will be performed to provide the effective concentration at 50% (EC50) of maximal basophil activation.

The following indicators of immune tolerance status can be examined from the assay of serum isolated from one or more blood samples obtained from the subject: Ratio of peanut specific IgE to IgG as measured by ImmunoCap assay.

In combination, results from the above analyses can be used to determine an immune tolerance signature and whether the subject has maintained immunological tolerance or not. If such analyses indicate weakening and/or loss of immunological tolerance, TIMP-PPE may be re-administered to the subject to restore immunological tolerance.

For example, the proportion of Th2a+ cells at the pre-dose Day 1 timepoint are expected to be >15% in peanut allergic subjects. Treatment with TIMP-PPE is expected to reduce the proportion of Th2a+ cells to <15% 14 days after the second dose indicative of induction of immunological tolerance. Increase in the proportion of Th2a+ cells to >15% at any of the subsequent timepoints (e.g., Days 90, 180, 270, and 360 post-dose) would be indicative of weakening of immunological tolerance and warrant re-administration of TIMP-PPE for restoration of immunological tolerance.

For example, the ratio of IL-5 to IFN-γ in the PBMC culture supernatant following ex vivo stimulation with peanut antigen proteins at Day 14 after treatment with TIMP-PPE is expected to be significantly decreased compared to the ratio at Baseline prior to treatment with TIMP-PPE. A statistically significant increase (e.g., by >10% or >1.5-fold) in the ratio of IL-5 to IFN-γ at any of the subsequent sampling and assay timepoints (e.g., Days 90, 180, 270, and 360 post-dose) would be indicative of weakening and/or loss of immune tolerance and warrant re-administration of TIMP-PPE for restoration of immune tolerance.

For example, the ratio of peanut-specific IgE to IgG in the blood of a subject treated with TIMP-PPE is expected to be significantly reduced at Day 60 post-treatment compared to the ratio of peanut-specific IgE to IgG at Baseline prior to treatment with TIMP-PPE. A statistically significant increase (e.g., by >10% or >1.5-fold) in the ratio of peanut-specific IgE to IgG at any subsequent sampling and assay timepoints (e.g., Days 90, 180, 270, and 360 post-dose) would be indicative of weakening and/or loss of immune tolerance and warrant re-administration of TIMP-PPE for restoration of immune tolerance).

### Example 2

It is contemplated that maintenance of immunological tolerance may be monitored in a subject suffering from type-1 diabetes (T1D) who is treated or about to undergo treatment with antigen-specific tolerizing therapy consisting of TIMPs encapsulating T1D antigens (TIMP-T1D). Subjects are expected to receive two doses of TIMP-T1D one-week apart on Days 1 and 8.

Briefly, the immune tolerance status of the subject may be determined by obtaining one or more whole blood samples from the subject pre-dose on the day of the first TIMP-T1D administration (Day 1), 14 days after administration of the second dose, and then at every 90 days post-second dose (e.g., Days 90, 180, 270, and 360 post-second dose). Whole blood can then be processed to isolate PBMCs, basophils, neutrophils, plasma, and serum for downstream analyses.

The following indicators of immune tolerance status can be examined from assay of PBMCs isolated from one or more blood samples collected from the subject and stimulated ex vivo with purified T1D antigenic proteins:
a. Frequency of Treg/Tr1 cells (CD4+/CD25+/FoxP3+/Helios+/IL-10+) in PBMCs as determined by flow cytometry.
b. Frequency of PD-L1+ macrophages in PBMCs as determined by flow cytometry.
C. Frequency of CD206+ macrophages in PBMCs as determined by flow cytometry.
d. Level of IL-10 produced by PBMCs stimulated ex vivo with T1D antigens.
e. Level of IFN-γ produced by PBMCs stimulated ex vivo with T1D antigens.

The following indicators of immune tolerance status can be examined from the assay of serum isolated from one or more blood samples obtained from the subject: Levels of T1D antigen-specific autoantibodies.

In combination, results from the above analyses can be used to determine an immune tolerance signature and whether the subject has maintained immunological tolerance or not. If such analyses indicate weakening and/or loss of immunological tolerance, TIMP-T1D may be re-administered to the subject to restore immunological tolerance.

For example, the frequency of Treg/Tr1 cells at the pre-dose Day 1 timepoint is expected to be approximately 1% in T1D subjects. Treatment with TIMP-T1D is expected to result in an increase in the frequency of Treg/Tr1 cells to 2-5% 14 days post-second dose indicating induction of immunological tolerance. Decrease in the frequency of Treg/Tr1 cells to 1% or lower at any of the subsequent timepoints (e.g., Days 90, 180, 270, and 360 post-dose) would be indicative of weakening of immunological tolerance and warrant re-administration of TIMP-T1D for restoration of immunological tolerance.

The frequency of PD-L1+ and CD206+ macrophages in PBMCs are expected to be <1% in the pre-dose sample on Day 1. Treatment with TIMP-T1D is expected to induce an increase in the frequency of PD-L1+ and CD206+ macrophages to approximately 5-10% 14 days post-second dose. Decrease in the frequency of PD-L1+ and CD206+ to <2% at any of the subsequent timepoints (e.g., Days 90, 180, 270, and 360 post-dose) would be indicative of weakening of immunological tolerance and warrant re-administration of TIMP-T1D for restoration of immunological tolerance.

Ex vivo stimulation of PBMCs with T1D antigens is expected to induce a 2-10-fold induction in the levels of IL-10 in samples collected 14 days post-second dose when compared to the pre-dose Day 1 sample indicating induction of immunological tolerance. Reduction in IL-10 production from PBMCs collected at any of the subsequent post-dose timepoints stimulated ex vivo with T1D antigens by 2-10-fold compared to the sample collected 14 days post-second dose would indicate weakening and/or loss of immunological tolerance warranting re-administration of TIMP-T1D.

Ex vivo stimulation of PBMCs with T1D antigens is expected to result in a 2-10-fold reduction in the levels of IFN-γ in samples collected 14 days post-second dose when compared to the pre-dose Day 1 timepoint indicating induction of immunological tolerance. Increased IFN-γ production from PBMCs collected at any of the subsequent post-dose timepoints stimulated ex vivo with T1D antigens by 2-10-fold compared to the sample collected 14 days post-second dose would indicate weakening and/or loss of immunological tolerance warranting re-administration of TIMP-T1D.

Treatment with TIMP-T1D is expected to result in a 2-10-fold reduction in the levels of T1D specific autoantibodies in serum samples collected 14 days post-second dose when compared to the pre-dose Day 1 sample indicating successful induction of immune tolerance. Increase in the levels of T1D specific autoantibodies in serum samples collected at any of the subsequent timepoints by 2-4-fold compared to the levels determined from the serum sample collected 14 days post-second dose would indicate weakening and/or loss of immunological tolerance warranting re-administration of TIMP-T1D.

### Example 3

It is contemplated that maintenance of immunological tolerance may be monitored in a subject suffering from Primary Biliary Cholangitis (PBC) who is treated or about to undergo treatment with antigen-specific tolerizing therapy consisting of TIMPs encapsulating PBC antigen (TIMP-PBC). Subjects are expected to receive two doses of TIMP-PBC one-week apart on Days 1 and 8.

Briefly, the immune tolerance status of the subject may be determined by obtaining one or more whole blood samples from the subject pre-dose on the day of the first TIMP-PBC administration (Day 1), 14 days after administration of the second dose, and then at every 90 days post-second dose (e.g., Days 90, 180, 270, and 360 post-second dose). Whole blood can then be processed to isolate PBMCs, basophils, neutrophils, plasma, and serum for downstream analyses.

The following indicators of immune tolerance status may be assayed from PBMCs stimulated ex vivo for 12-14 hours with PBC disease associated antigen such as the PDC-E2160-175 antigenic epitope in combination with anti-CD40 antibody:
a. Frequency of CD4+T effector cells (CD4+CD154+CD137+).
b. Frequency of antigen specific CD8+ Teff (CD8+CD69+CD137+).
c. Frequency of antigen specific Treg cells (CD4+CD25+CD127-CD154-CD137+GARP+/−).

The following indicators of immune tolerance status can be examined from the assay of serum isolated from one or more blood samples obtained from the subject: Levels of anti-mitochondrial antibodies.

Results from the analysis of the above parameters assessed from the pre-dose and each post-dose sample may be compared to determine whether the subject has maintained immunological tolerance. If such analyses indicate weakening and/or loss of immunological tolerance, TIMP-T1D may be re-administered to the subject to restore immunological tolerance.

It is expected that the frequency of CD4+T effector cells in ex vivo stimulated PBMC cultures from samples collected prior to treatment of TIMP-PBC will be approximately 20-30%. Treatment with TIMP-PBC is expected to reduce the frequency of CD4+T effector cells to approximately 10-12% in samples collected 14 days post-second dose of TIMP-PBC. Increase in the frequency of CD4+T effector cells to 15%-20% in samples obtained at subsequent timepoints would be indicative of weakening of immunological tolerance and warrant re-administration of TIMP-PBC for restoration of immunological tolerance.

It is expected that the frequency of antigen specific CD8+ Teff in ex vivo stimulated PBMC cultures from samples collected prior to treatment of TIMP-PBC will be approximately 30-35%. Treatment with TIMP-PBC is expected to reduce the frequency of CD8+T effector cells to approximately 10-15% in samples collected 14 days post-second dose of TIMP-PBC. Increase in the frequency of CD8+T effector cells to 15%-20% in samples obtained at subsequent timepoints would indicative of weakening of immunological tolerance and warrant re-administration of TIMP-PBC for restoration of immunological tolerance.

It is expected that the frequency of antigen specific Treg cells in ex vivo stimulated PBMC cultures from samples collected prior to treatment of TIMP-PBC will be approximately 1-2%. Treatment with TIMP-PBC is expected to increase the frequency of antigen specific Treg cells to approximately 5-10% in samples collected 14 days post-second dose of TIMP-PBC. Decrease in the frequency of antigen specific Treg cells to <2% in samples obtained at subsequent timepoints would indicative of weakening of immunological tolerance and warrant re-administration of TIMP-PBC for restoration of immunological tolerance.

Additional analyses of T cells may be performed by assay of cell surface markers (CD4, CD45RA), maturation markers (CCR7, CD27), markers of in vivo activation (CD38), markers of in vitro activation (CD69, GARP, OX40), exhaustion markers (TIGIT, PD1, KLRG1) and chemokine receptors (CRTH2, CXCR5, CXCR3, CCR6, CCR4).

Treatment with TIMP-PBC is expected to result in a 2-10-fold reduction in the levels of anti-mitochondrial antibodies in serum samples collected 14 days post-second dose when compared to the pre-dose Day 1 sample indicating successful induction of immune tolerance. Increase in the levels of anti-mitochondrial specific antibodies in serum samples collected at any of the subsequent timepoints by 2-4-fold compared to the levels determined from the serum sample collected 14 days post-second dose would indicate weakening and/or loss of immunological tolerance warranting re-administration of TIMP-PBC.

Numerous modifications and variations in the invention as set forth in the above illustrative examples are expected to occur to those skilled in the art. Consequently only such limitations as appear in the appended claims should be placed on the invention.

What is claimed:

1. A method for monitoring immune tolerance status of a subject undergoing immune tolerizing treatment for a disease or condition, the method comprising the steps of
(a) obtaining one or more biological samples from the subject prior to administration of treatment and determining the immune tolerance status of the subject by assaying said biological sample(s),
(b) obtaining one or more biological samples from the subject after administration of treatment and determining the immune tolerance status of the subject by assaying the biological sample(s), and
(c) obtaining one or more biological samples from the subject at regular intervals after administration of treatment and determining the immune tolerance status of the subject by assaying the biological sample(s),
(d) comparing the results from the assay of one or more biological samples in step (c) with the results of steps (a) and/or (b) to generate an immune tolerance signature, and
(e) re-administering the tolerizing treatment if the immune tolerance signature indicates a weakening, and/or loss of immunological tolerance.

2. The method of claim 1, wherein the disease or condition is selected from the group consisting of an inflammatory condition, an autoimmune condition, an allergy, an abnormal immune response, a lysosomal storage disease, an enzyme deficiency, a protein deficiency, a genetic disorder, and/or is a transplant recipient.

3. The method of claim 2, wherein the autoimmune condition is selected from the group consisting of atopic atopic dermatitis, multiple sclerosis, autoimmune myelitis, myelitis, transverse myelitis, neuromyelitis optica (NMO), neuromyelitis optica spectrum disorder (NMSOD), type-1 diabetes (T1D), type-2 diabetes (T2D), Celiac Disease (CD), Grave's Disease, Myasthenia Gravis, acute disseminated encephalomyelitis, Addison's Disease, alopecia, rheumatoid arthritis, osteoarthritis, ankylosing spondylitis, autoimmune myocarditis, autoimmune neutropenia, autoimmune skin disease, autoimmune uveitis, ballus pemphigoid, Behcet's Syndrome, cerebral degeneration, chronic neuropathy, cicatrical pemphigoid, pemphigus vulgaris, Crohn's Disease, Inflammatory Bowel Disease (IBD), Inflammatory Bowel Syndrome (IBS), cryopathy, dermatitis hyperformis, Eaton Lambert's Disease, encephalomyelitis, epidermolysis bullosa acquisita, erythema nodosa, glomerulonephritis, Goodpasture's Disease, granulomatosis, Guillain-Barre Syndrome, Hashimoto Disease, Kawasaki Disease, hemolytic anemia, hypersensitivity vasculitis, lupus erythematosus, mixed connective tissue disease, mixed essential cryoglobulinemia, multifocal motor neuropathy, opsonoclonus-myoclonus, paraneoplastic pemphigus, pemphigoig gestationis, pemphigus folaceus, pernicious anemia, peripheral biliary cirrhosis, polyangiitis overlap syndrome, polyarteritis nodosa, polyglandular failure, polyglandular syndrome, polymyositis/dermatomyositis, psoriasis, eczema, retinopathy, Reynaud's Syndrome, sarcoidosis, Scleroderma Type 1, sclerosis cholangitis, Sjogren's Syndrome, Stiffman's syndrome, Takayasu arteritis, termporal arteritis, thyroiditis, ulcerative colitis, immune thrombocytopenia purpura, thrombotic thrombocytopenia purpura, autoimmune hepatitis, primary biliary cholangitis (PBC), ANCA diseases, Granulamatosis with Polyangiitis, and Microscopic Polyangiitis.

4. The method of claim 1 or 2, wherein the immune tolerizing treatment comprises an antigen selected from the group consisting of one or more autoimmune antigens, allergens, enzyme replacement therapies, protein therapies, transplant antigens, and gene therapy vectors.

5. The method of claim 4, wherein the one or more autoimmune antigens are selected from the group consisting of myelin basic protein, acetylcholine receptor, endogenous antigen, myelin oligodendrocyte glycoprotein (MOG), myelin basic protein (MBP), proteolipid protein (PLP), myelin associated glycoprotein (MAG), cyclic nucleotide phosphohydrolase, pancreatic beta-cell antigen, insulin, proinsulin, islet-specific glucose-6-phophatase catalytic subunit-related protein (IGRP), glutamic acid decarboxylase (GAD), collagen type 11, human cartilage gp39, fp130-RAPS, fibrillarin, small nucleolar protein, thyroid stimulating factor receptor, histones, glycoprotein gp70, pyruvate dehydrogenase dehydrolipoamide acetyltransferase (PCD-E2), hair follicle antigen, aqua porin 4, Desmoglein 1, Desmoglein 3, nicotinic acetylcholine receptor, gliadin, ADAMTS13, GPIlb/GPIlla, CYP2D6, BP180, NC16, BP230, Ro60, MPO, thyroid stimulating hormone receptor, and human tropomyosin isoform 5.

6. The method of claim 4, wherein the one or more allergens is selected from the group consisting of Bahia grass pollen (BaGP), peach allergen, milk allergens, celery allergens, nut allergens, bovine serum albumin, Hazelnut allergens, ovalbumin, egg allergen, peanut allergens, fish allergens, shellfish allergens, and Japanese cedar pollen.

7. The method of any one of claims 1-6, wherein said immune tolerizing treatment is antigen specific.

8. The method of any one of claims 1-7, wherein the immune tolerizing treatment is selected from the group consisting of oral immunotherapy (OIT), subcutaneous immunotherapy (SCIT), sublingual immunotherapy (SLIT), and immune tolerizing nanomedicine.

9. The method of claim 8, wherein the immune tolerizing nanomedicine comprises tolerizing immune modifying particles (TIMPs).

10. The method of any one of claims 1-9, wherein said biological sample(s) of any one of steps (a)-(c) are obtained 1-7 days, 1-4 weeks, and/or 1-12 months prior to or after administration of the treatment.

13. The method of any one of claims 1-12, wherein the one or more biological samples are selected from the group consisting of whole-blood, peripheral blood, peripheral blood mononuclear cells (PBMCs), serum, plasma, urine, cerebrospinal fluid (CSF), stool, a tissue biopsy, and a bone-marrow biopsy.

14. The method of any one of claims 1-13, wherein said assay of the one or more biological samples is selected from the group consisting of analyzing cells, cell-surface proteins, extracellular proteins, intracellular proteins, nucleic acids, metabolites, and combinations thereof.

15. The method of claim 14, wherein the cells are immune cells and/or non-immune cells.

16. The method of claim 15, wherein the immune cells are innate immune cells and/or adaptive immune cells.

17. The method of claim 15 or 16, wherein the immune cells are selected from the group consisting of monocytes, macrophages, neutrophils, granulocytes, dendritic cells, mast cells, eosinophils, basophils, T-cells, B-cells, NK cells, and NK-T cells.

18. The method of claim 17, wherein the T cells are T1 cells, T2a cells, Treg cells, Tr1 cells and/or Teff cells.

19. The method of claim 15, wherein the non-immune cells are selected from the group consisting of epithelial cells, stromal cells, endothelial cells, fibroblasts, pericytes, adipocytes, mesenchymal stem cells, hematopoietic stem cell, hematopoietic progenitor cells, liver sinusoidal endothelial cells (LSECs), and/or Kupffer cells.

20. The method of any one of claims 1-19, wherein the assay of one or more biological samples in Step (a) is used to determine the immune tolerance status of the subject at Baseline.

22. The method of claim 21, wherein the immune tolerance status of the subject at Baseline is determined by comparing the results from the assay of one or more biological samples in Step (a) to the assay of samples obtained from a healthy subject.

23. The method of claim 20 or 21, wherein the immune tolerance status of the subject at Baseline is used to determine whether the subject is administered a tolerizing treatment.

24. The method of any one of claims 20-23, wherein the immune tolerance status of the subject at Baseline indicates weak immune tolerance or absence of immune tolerance.

25. The method of any one of claims 20-24, wherein the subject is administered treatment if the immune tolerance status of the subject at Baseline indicates weak immune tolerance or absence of immune tolerance.

26. The method of any one of claims 1-25, wherein the subject is re-administered treatment in Step (e) if the results of an assay in Step (c) compared to the results from the assay in Steps (a) and/or (b) indicate weakening and/or loss of immune tolerance.

27. The method of any one of claims 1 to 26, wherein the immune tolerance signature of a subject is generated using one or more of the following parameters assayed from one or more biological samples obtained from the subject and stimulated in vivo and/or ex vivo:
a. proportion of effector T cells in the total T cell population,
b. proportion of Treg cells in the total T cell population,
c. proportion of effector B cells in the total B cell population,
d. levels and/or ratios of specific IgG, IgA, IgM, and/or IgE,
e. levels of inflammatory cytokines and chemokines,
f. levels of anti-inflammatory cytokines and chemokines,
g. levels of inflammatory metabolites, and/or
h. levels of anti-inflammatory metabolites.

28. The method of claim 27, wherein the immune tolerance signature is indicative of maintenance of immune tolerance if 2, 3, 4, 5, 6, 7, or 8 parameters listed in (a)-(h) indicate maintenance of immune tolerance. [

29. The method of claim 28, wherein the subject is determined to not require treatment with TIMPs if at least 2/8 parameters listed in (a)-(h) above indicate maintenance of immune tolerance.

30. The method of any one of claims 1-29, wherein the subject is re-administered treatment if immune tolerance is weakened and/or lost by about 5%-100% in at least one parameter of immune tolerance.

31. The method of claim 30 wherein the at least one parameter of immune tolerance is selected form the group consisting of:
a. proportion of effector T cells in the total T cell population,
b. proportion of Treg cells in the total T cell population,
c. proportion of effector B cells in the total B cell population,
d. levels and/or ratios of specific IgG, IgA, IgM, and/or IgE,
e. levels of inflammatory cytokines and chemokines,
f. levels of anti-inflammatory cytokines and chemokines,
g. levels of inflammatory metabolites, and/or
h. levels of anti-inflammatory metabolites,
assayed from one or more biological samples obtained from the subject and stimulated in vivo and/or ex vivo.

32. The method of claim 30, wherein immune tolerance is weakened and/or lost by 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%.

33. The method of any one of claims 1-29, wherein the subject is re-administered treatment in Step (e) if the results of Step (d) indicate weakening and/or loss of immune tolerance by about 2-100-fold in at least one parameter of immune tolerance.

34. The method of claim 33 wherein the at least one parameter of immune tolerance is selected form the group consisting of:
a. proportion of effector T cells in the total T cell population,
b. proportion of Treg cells in the total T cell population,
c. proportion of effector B cells in the total B cell population,
d. levels and/or ratios of specific IgG, IgA, IgM, and/or IgE,
e. levels of inflammatory cytokines and chemokines,
f. levels of anti-inflammatory cytokines and chemokines,
g. levels of inflammatory metabolites, and/or
h. levels of anti-inflammatory metabolites,
assayed from one or more biological samples obtained from the subject and stimulated in vivo and/or ex vivo.

35. The method of claim 33 or 34, wherein immune tolerance is weakened and/or lost by about 2, 5, 10, 15, 20, 25, 30, 35, 40, 45, 50, 55, 60, 65, 70, 75, 80, 85, 90, 95, or 100-fold.

36. The method of any one of claims 1-35, wherein the treatment administered in Step (e) is TIMPs.

37. The method of claim 36, wherein the TIMPs are administered at a dose level of between about 0.1 to 12 mg/kg.

38. The method of claim 37, wherein the TIMPs are administered at a dose level of about 0.1 mg/kg, 0.25 mg/kg, 0.5 mg/kg, 0.75 mg/kg, 1.0 mg/kg, 1.25 mg/kg, 1.5 mg/kg, 2.0 mg/kg, 2.5 mg/kg, 3 mg/kg, 3.5 mg/kg, 4.0 mg/kg, 4.5 mg/kg, 5 mg/kg, 5.5 mg/kg, 6 mg/kg, 6.5 mg/kg, 7 mg/kg, 7.5 mg/kg, 8.0 mg/kg, 8.5 mg/kg, 9 mg/kg, 9.5 mg/kg, 10 mg/kg, 10.5 mg/kg, 11 mg/kg, 11.5 mg/kg, or 12 mg/kg.

39. The method of claim 36, wherein the TIMPs are administered at a dose level of between about 50 mg to 800 mg.

40. The method of claim 39, wherein the TIMPs are administered at a dose level of about 50 mg, 75 mg, 100 mg, 125 mg, 150 mg, 175 mg, 200 mg, 225 mg, 250 mg, 275 mg, 300 mg, 325 mg, 350 mg, 375 mg, 400 mg, 425 mg, 450 mg, 500 mg, 525 mg, 550 mg, 575 mg, 600 mg, 625 mg, 650 mg, 675 mg, 700 mg, 725 mg, 750 mg, 775 mg, or 800 mg.

41. The method of any one of claims 36-40, wherein the TIMPs are administered in a single dose or multiple doses.

42. The method of any one of claims 36-41, wherein the TIMPs are administered once weekly, once every two weeks, once every three weeks, once every 4 weeks, once every two months, once every three months, once every 6 months, or once per year.

43. The method of any one of claims 36-42, wherein the TIMPs are administered intravenously, subcutaneously, intramuscularly, intraperitoneally, intranasally, or orally.

44. The method of any one of claims 1-43, wherein said tolerance status is determined by assay of one or more biological samples after one or more stimuli.

45. The method of claim 44, wherein the stimuli are provided in vivo and/or ex vivo.

46. The method of claim 44 or 45, wherein the one or more stimuli are selected from the group consisting of one or more antigens, allergens, and activating agents.

47. The method of claim 46, wherein the antigens and allergens are associated with the disease or condition being treated.

48. The method of claim 47, wherein the antigens and allergens are not associated with the disease or condition being treated.

49. The method of claim 46, wherein the one or more activating agents are selected from the group consisting of an antibody, a chemical agent, a viral component, and a bacterial component.
